(19) United States
(12) Patent Application Publication
(10) Pub. No.: US 2016/0041171 A1
(43) Pub. Date: Feb. 11, 2016

(54) SYSTEMS AND METHODS FOR FACILITATING DIAGNOSIS, PROGNOSIS AND TREATMENT OF CANCER BASED ON DETECTION OF HER3 ACTIVATION

(71) Applicant: LABORATORY CORPORATION OF AMERICA HOLDINGS, Burlington, NC (US)

(72) Inventors: Gerald J. Wallweber, Foster City, CA (US); John W. Winslow, El Granada, CA (US); Lisa DeFazio Eli, Redwood City, CA (US)

(21) Appl. No.: 14/781,468

(22) PCT Filed: Apr. 7, 2014
(86) PCT No.: PCT/US2014/033208

§ 371 Date: Sep. 30, 2015

Related U.S. Application Data
(60) Provisional application No. 61/809,083, filed on Apr. 5, 2013.

(51) Int. Cl. G01N 33/574; A61K 45/06

(52) U.S. Cl. CPC G01N 33/57415; A61K 45/06; G01N 2333/912; G01N 2333/71; G01N 2440/14; G01N 2333/9121

(57) ABSTRACT

Systems and methods are provided for facilitating diagnosis, prognosis and treatment of cancer based on detection of HER3 activation. The systems and methods involve analysis of samples for the presence or the absence of activated HER3 markers as indicated by HER2-HER3 heterodimers, HER3 phosphorylation, or recruitment of PI3K to activated HER3 (HER3/PI3K complexes). The amounts of activated HER3 marker expression may be measured alone or in conjunction with other biomarkers.

### CROSS-REFERENCE TO RELATED APPLICATIONS

This application claims the benefit of and priority under 35 U.S.C. §119(e) to U.S. Provisional Application No. 61/809,083, filed Apr. 5, 2013, which is incorporated herein by reference in its entirety.

### FIELD OF THE INVENTION

The present invention relates generally to systems and methods for facilitating diagnosis, prognosis and treatment of cancer based on detection of HER3 activation.

### BACKGROUND OF THE INVENTION

Certain members of the tyrosine kinase receptor superfamily that are associated with cell proliferation, survival, and migration are the human epidermal growth factor receptors or HER protein family. These HER proteins include HER1 (also known as EGFR and ErbB1), HER2 (ErbB2), HER3 (ErbB3), and HER4 (ErbB4). Expression levels of each of these individual cell surface receptors have been evaluted as cancer biomarkers.

Both ligand-induced and ligand independent dimerization and activation of HER receptors are known to occur, including formation of the HER2-HER3 heterodimer in HER2 amplified cells. Dimerization is followed by receptor transactivation and phosphorylation, the recruitment of various cytosolic proteins to the phosphorylated receptors, thereby triggering various signaling cascades including the phosphatidylinositol 3-kinase (PI3K)/Akt, PKC, MAPK and the Ras signaling pathways.

HER3 is a unique member of the ErbB receptor family. Unlike HER1 and HER2, it cannot form a homodimer and lacks the intracellular kinase activity. Although it has weak tyrosine kinase activity, HER3 is generally considered an inactive “pseudokinase.” However, the C-terminal region of HER3 contains six consensus phosphotyrosine sites which bind the SH2 domain of PI3K, implicating its crucial role in the activation of the PI3 K/Akt pathway. HER3 is also a mechanism by which HER signaling activity can be activated despite significant inhibition of other ErbB kinases. Sergina, N. V., et al., Nature 445 (7126):437-441 (2007). This unique capability of HER3 is not noted in any other HER protein. Recent studies showed that expression and translocation of HER3 from the nucleus to the membrane were also responsible for resistance to EGFR or HER2 targeted therapy. Sergina, N. V., et al., Nature 445 (7126):437-441 (2007); Hsieh, A. C. and Moasser, M. M., Brit. J. Cancer 97(4):453-457 (2007).

Upregulation of HER3 is commonly seen in various malignancies such as breast cancer, colorectal carcinoma, squamous cell carcinoma of the head and neck (SCCHN), uveal melanoma, and gastric, ovarian, prostate, and bladder cancers. See generally, Jiang, N., Chemother. Res. Pract. 2012:id817304. In human breast cancers, both HER3 mRNA and protein are upregulated. Compared to normal breast tissue, HER3 protein overexpression has been reported in 50-70% of human breast cancers and seems to be associated with metastasis, tumor size, and risk of local recurrence. Increased HER3 mRNA or protein is commonly seen in tumors such as colon carcinomas and is associated with lymph node metastasis and a shorter time to progression. In SCCHN, a high HER3 expression seems to be associated with increased metastasis and decreased overall survival. Moreover, HER3 expression is correlated with resistance to the HER1 inhibitor gefitinib in SCCHN. This suggests that HER3 expression plays a significant role in carcinogenesis and would be a reasonable target for anticancer therapy. Because its functions are highly dependent on heterodimerization with other members, HER3 cannot transform cells through overexpression or mutational activation. Its primary binding partner is HER2, and it plays an important role in HER2 transforming and accelerating progression in human cancers. In addition, HER3 has also been identified as a potential marker of drug sensitivity. See generally, Mukherjee, A., et al., PLOS One 6(1):e16443 (2011),

Despite advances in the development of drug therapies targeted to HER1 and HER2, mechanisms for resistance are well-documented. See generally, Jiang, N., Chemother. Res. Pract. 2012:id817304. Specifically, compensatory HER3 signaling and sustained PI3K/Akt activation have been implicated as playing an important role in the resistance to HER-targeted therapy. Potential mechanisms for this resistance include upregulated HER3 expression at the membrane, increased HER3 ligand heregulin expression, and cooperation with other receptor tyrosine knases (e.g., MET). Both HER3 inhibitors and pan-ErbB inhibitors, which simultaneously inhibit HER3 and other family members, have been developed and a number of them are in clinical development. Most HER3 inhibitors under development target the extracellular domain of the protein. As HER3 lacks enzymatic catalytic activity, its functions cannot be inhibited using tyrosine kinase inhibitors. In addition to monoclonal antibodies, inhibition of HER3 dimerization with other ErbB family members is a valid approach. Pertuzumab targets the dimerization interface of HER2 thus disrupts ligand-induced HER2-HER3 dimerization.

As HER3-targeted therapies are showing promising results in pre-clinical studies, it would be useful to assess whether an individual has high levels of activated HER3, and thus is an appropriate candidate for HER3-acting agents. No effective method of doing so has been developed. It is with this limitation in mind that the present invention was developed.

### BRIEF SUMMARY OF THE INVENTION

In one aspect, the present invention provides systems and methods for facilitating diagnosis, prognosis, and treatment of cancer by detecting HER3 activation.

In another aspect, the present invention provides methods for measuring the amount of activated HER3 in a tumor, comprising: (a) measuring in a tumor sample the amount of total HER3 and the amount of at least one of HER2-HER3 heterodimer, phosphorylated HER3, or HER3/PI3K complex in the sample; (b) determining the ratio of at least one of HER2-HER3 heterodimer, phosphorylated HER3, or HER3/PI3K complex to total HER3 protein; and (c) indicating that the tumor has a high amount of activated HER3 if (i) the amount of total HER3 in the sample is above the median amount of total HER3 of a reference population and (ii) the ratio of at least one of HER2-HER3 heterodimer to HER3 total, phosphorylated HER3 to total HER3 in the sample, or HER3/PI3K complex is above the median ratio of at least one of HER2-HER3 heterodimer to HER3 total, phosphorylated HER3 to total HER3, or or HER3/PI3K complex to total HER3 in the reference population.

In another aspect, the present invention provides methods of treating a subject with cancer comprising: (a) measuring in a tumor sample from a subject the amount of total HER3 protein and at least one of HER2-HER3 heterodimer, phosphorylated HER3, or HER3/PI3K complex; (b) determining the ratio of the amount of at least one of HER2-HER3 heterodimer, phosphorylated HER3, or HER3/PI3K complex to total HER3; (c) determining if a subject has a cancer characterized as having a high level of activated HER3, wherein a high level of activated HER3 comprises (i) the amount of total HER3 in the sample being above the median amount of total HER3 of a reference population of subjects having the same type of cancer as the subject and (ii) the ratio of at least one of the amount of HER2-HER3 homodimer to the amount of HER3 total, the amount of phosphorylated HER1 to the amount of total HER3, or the amount of HER3-PI3K complex to the amount of HER3 total in the sample being above the median ratio of at least one of the amount of HER2-HER3 homodimer to the amount of HER3 total, the amount of phosphorylated HER3 to the amount of total HER3, or the amount of HER3-PI3K complex to the amount of HER3 total in the reference population of subjects having the same type of cancer as the subject; and (d) administering a HER3-targeted therapy to the subject if the subject has a cancer characterized as having a high level of activated HER3.

In another aspect, the present invention provides methods for predicting responsiveness of a subject with cancer to a HER3 acting agent comprising: (a) measuring in a tumor sample from a subject the amount of total HER3 protein and at least one of HER2-HER3 heterodimer, phosphorylated HER3, or HER3/PI3K complex; (b) determining the ratio of the amount of at least one of HER2-HER3 heterodimer, phosphorylated HER3, or HER3/PI3K complex to total HER3; (c) determining if a subject has a cancer characterized as having a high level of activated HER3, wherein a high level of activated HER3 comprises (i) the amount of total HER3 in the sample being above the median amount of total HER3 of a reference population of subjects having the same type of cancer as the subject and (ii) the ratio of at least one of the amount of HER2-HER3 homodimer to the amount of HER3 total, the amount of phosphorylated HER1 to the amount of total HER3, or the amount of HER3-PI3K complex to the amount of HER3 total in the sample being above the median ratio of at least one of the amount of HER2-HER3 homodimer to the amount of HER3 total, the amount of phosphorylated HER3 to the amount of total HER3, or the amount of HER3-PI3K complex to the amount of HER3 total in the reference population of subjects having the same type of cancer as the subject; and (d) indicating that the subject is more likely to respond to the HER3 acting agent if the subject's cancer is characterized as having a high level of activated HER3.

In another aspect, the invention comprises systems comprising a first computing device, the first computing device in communication with a database; a first application executing on the first computing device, the first application configured to receive a plurality of laboratory test results for a plurality of subjects and store the plurality of laboratory test results in the database, wherein the plurality of laboratory test results comprise an amount of total HER3 and at least one of an amount of HER2-HER3 heterodimer, phosphorylated HER3, or HER3/PI3K complex measured in a tumor sample from a subject; a second computing device, the second computing device in communication with the database; and a second application executing on the second computing device, the second application configured to query the database for laboratory test results for a subject from the plurality of subject; receive the laboratory test results for the subject from the database; determine a test result based at least in part on the received laboratory test results for the subject, the test results comprising the amount of total HER3 and the ratio of at least one of HER2-HER3 heterodimer, phosphorylated HER3, or HER3/PI3K complex to total HER3 protein in a tumor sample obtained from the subject; generate a test result report for the subject, the test result comprising the amount of activated HER3 in the tumor sample and based at least in part on the test result for the subject; and transmit the test result report for the patient to a third computing device.

In another aspect, the invention comprises methods comprising receiving a plurality of laboratory test results in a database, wherein the plurality of laboratory test results comprise an amount of total HER3 and at least one of an amount of HER2-HER3 heterodimer, phosphorylated HER3, or HER3/PI3K complex measured in a tumor sample from a subject; storing the plurality of laboratory test results in the database; querying the database for laboratory test results for a subject from the plurality of subjects; receiving the laboratory test results for the subject from the database; determining a test result based in part on the received laboratory test results for the subject, the test result comprising the amount of total HER3 and the ratio of at least one of HER2-HER3 heterodimer, phosphorylated HER3, or HER3/PI3K complex to total HER3 protein in a tumor sample obtained from the subject; generating a test result report for the subject, the test result report comprising the amount of activated HER3 in the tumor sample and based at least in part on the test result for the subject; and transmitting the test result report for the subject to a computing device.

These illustrative embodiments are mentioned not to limit or define the invention, but rather to provide examples to aid understanding thereof. Illustrative embodiments are discussed in the Detailed Description, which provides further description of the invention. Advantages offered by various embodiments of this invention may be further understood by examining this specification.

### BRIEF DESCRIPTION OF THE DRAWINGS

The present invention may be better understood by reference to the following non-limiting figures.

FIG. 1 is a schematic diagram showing schematic representations of various VeraTag® assay formats in accordance with certain embodiments of the invention. Assay formats are shown for detection of HER2-HER3 heterodimers for exemplary purposes; the assay can be modified as described herein for detection of other targets. Panels A and B show a light release format, while Panel C shows a reducing (DTT) format. Panel A depicts analysis of formalin-fixed paraffin-embedded (FFPE) tissue samples, and Panel B depicts analysis of a tissue lysate sample. In the light release format, diffusing reactive singlet oxygen may be used to cleave the covalent linker between a VeraTag® reporter molecule and a HER3 antibody in response to photo-induction of the cleavage-inducing agent by light. The reactive singlet oxygen is generated from the cleaving probe attached to a HER2 antibody. In the reducing format, a reducing agent is used to induce cleavage of the covalent linker between a VeraTag® reporter molecule and a HER3 antibody. Following cleavage, capillary electrophoretic (CE) separation of the VeraTag® reporter molecules may be conducted and assessed by electropherogram. The x-axis shows the time at which the cleaved VeraTag® reporter molecule eluted from the capillary, and the fluorescence intensity is shown on the y-axis. Fluorescent peaks v1 and v2 denote the elution of two different VeraTag® reporter molecules. “HER3” represents a HER3 monomer; “HER2” represents a HER2 monomer; “B” represents a biotin molecule; “S” represents a streptavidin molecule, “Tag” represents a VeraTag® reporter molecule, and “hv” represents light energy.

FIG. 2 is a series of graphs showing VeraTag® assay results for detection of certain biomarkers to determine consistency between batches of breast tumor samples assessed in accordance with certain embodiments of the invention. Cell line control sample data are shown on the left side of each graph and breast tumor sample data are shown on the right side of each graph. The fluorescence intensity for each sample is shown on the y-axis, measured in relative peak area (RPA). Panels A and B show results from assays measuring levels of total HER2 and total HER3, respectively. Panels C, D, and E show results from assays measuring HER2-HER3 heterodimers, HER3 phosphorylated at tyrosine 1289, and HER3/PI3K complexes, respectively.

FIG. 3 shows a scatterplot of HER2 total measurement as assessed by HERmark® Assay for 1090 breast cancer tissue specimens from three combined cohorts. The HERmark® assay measurements are compared to HER2 status (HER2 positive or HER2 negative) as determined using reference methods at a central testing lab (designated as “central HER2 positive” and “central HER2 negative”). This comparison was used to define the HERmark® status of a sample.

FIG. 4 shows scatter plots depicting the distribution of biomarker levels measured in FIG. 3 separated based on whether the sample was determined to be HER2 positive or HER2 negative based on HERmark® analysis in accordance with certain embodiments of the invention.

FIG. 5 shows graphs illustrating the statistical relationship between the markers measured in FIG. 3 in accordance with certain embodiments of the invention. Panel A shows the correlation between HER2-HER3 heterodimers and total HER2, Panel B shows the correlation between HER2-HER3 heterodimers and total HER3, and Panel C shows the correlation between HER2-HER3 heterodimers and phosphorylated HER3.

FIG. 6 shows graphs illustrating the statistical relationship between the markers measured in FIG. 3 in accordance with certain embodiments of the invention. Panel A shows the correlation between phosphorylated HER3 and total HER2, Panel B shows the correlation between phosphorylated HER3 and total HER3.

FIG. 7 shows pairwise comparison of the biomarker levels in the tumor samples in accordance with certain embodiments of the invention. Biomarker levels were measured using VeraTag® assays. Panel A shows the data for all of the assessed breast tumor samples. Panel B shows the data for the HERmark® negative (low HER2) tumor samples. Panel B shows the data for the HERmark® positive (high HER2) tumor samples. The Spearman correlation coefficients having significant p-values (p<0.05) are underlined.

FIG. 8 shows heat map diagrams illustrating biomarker levels for the tumor samples in accordance with certain embodiments of the invention. Biomarker levels were measured using VeraTag® assays. Panel A is a heat map for all of the breast tumor samples. Panel B shows two heat maps, one for HERmark® negative (low HER2) tumor samples (left) and one for HERmark® positive (high HER2) tumor samples (right). Samples are sorted by highest HERmark® HER2 levels (right) to the lowest (left) in each diagram. The sample number is indicated along the bottom of each heat map, and the biomarker analyzed in the assay is shown on the left side of each heat map. Samples that exhibited the highest expression (≧90^{th} percentile) are shown in dark grey; and samples with low expression (≦10^{th} percentile) are shown in light grey. Samples that exhibited medium expression (50^{th} percentile) are shown in black. The samples identified with an arrow are the samples that were considered to have the highest levels of activated HER3.

FIG. 9 shows a hierarchical cluster analysis of the breast tumor samples by biomarker level as measured by VeraTag® assays in accordance with certain embodiments of the invention. Panel A shows the analysis for samples characterized as HER2 negative by HERmark® analysis (low HER2). Panel B shows the analysis for samples characterized as HER2 positive by HERmark® analysis (high HER2). The sample numbers are shown on the bottom of each graph. The biomarkers analyzed are shown at the left of each graph: total HER2 (H2T); phosphorylated at tyrosine 1289 (p-HER3); HER2-HER3 heterodimers (H23D); total HER3 (H3T), and HER3/PI3K complex (HER3-PI3K). The tumors expressing the high levels of activated HER3 are shown in black or dark grey and correspond to the tumors expressing the high levels of activated HER3 as shown in the heat map analysis (FIG. 8).

FIG. 10 depicts a system diagram depicting exemplary computing devices in an exemplary computing environment according to certain embodiments of the invention.

FIG. 11 depicts a block diagram illustrating an operation of and systems and methods for a laboratory reporting system according to according to certain embodiments of the invention.

### DETAILED DESCRIPTION OF THE INVENTION

Embodiments of the present invention provide systems and methods for facilitating diagnosis, prognosis and treatment of cancer based on detection of HER3 activation. Certain embodiments involve measuring the levels of the amount of an activated HER3 protein in a biological sample from a subject having cancer.

### Definitions and Abbreviations

The term “about,” as used herein, unless otherwise indicated, refers to a value that is no more than 10% above or below the value being modified by the term. For example, the term “about 5 μg/kg” means a range of 4.5 μg/kg to 5.5 μg/kg. As another example, “about 1 hour” means a range of 48 minutes to 72 minutes.

As used herein, the term “activated HER3” refers to a molecular form of HER3 that is capable of initiating downstream signaling pathways. For example, activated forms of HER3 include HER2/HER3 heterodimers, phosphorylated HER3, and HER3 complexed with PI3K. For example, activated HER3 may be detected by measurement of HER2-HER3 heterodimer formation, phosphorylation of HER3, or recruitment of PI3K to an activated HER3 protein. Phosphorylated HER3 may be phosphorylated at the tyrosine residue at position 1289, or at one or more of several additional tyrosine residues. In addition, activated HER3 may be detected by detecting the recruitment and/or phosphorylation of other proteins that are associated with the activated HER3 protein (i.e., proteins downstream in the signaling pathway).

As used herein, the term “antibody” means an immunoglobulin that specifically binds to, and is thereby defined as complementary with, a particular spatial and polar organization of another molecule. The antibody can be monoclonal, polyclonal, or recombinant and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)₂, Fab′, and the like. Antibodies may also be single-chain antibodies or an antigen-binding fragment thereof, chimeric antibodies, humanized antibodies or any other antibody derivative known to one of skill in the art that retains binding activity that is specific for a particular binding site. In addition, aggregates, polymers, and conjugates of immunoglobulins or their fragments can be used where appropriate so long as binding affinity for a particular binding site is maintained. Guidance in the production and selection of antibodies and antibody derivatives for use in immunoassays, including such assays employing releasable molecular tag (as described below) can be found in readily available texts and manuals, e.g., Harlow and Lane, 1988, Antibodies: A Laboratory Manual, Cold Spring Harbor Laboratory Press, New York; Howard and Bethell, 2001, Basic Methods in Antibody Production and Characterization, CRC Press; Wild, ed., 1994, The Immunoassay Handbook, Stockton Press, New York.

“Antibody composition” refer to an antibody as defined above that is further modified by attachment to a label or other chemical moiety.

“Antibody binding composition” is used herein to refer to a molecule or a complex of molecules that comprises one or more antibodies, or antigen-binding fragments, that bind to a molecule, and derives its binding specificity from such antibody or antibody-binding fragment. Antibody binding compositions include, but are not limited to, (i) antibody pairs in which a first antibody binds specifically to a target molecule and a second antibody binds specifically to a constant region of the first antibody; a biotinylated antibody that binds specifically to a target molecule and a streptavidin protein, which protein is derivatized with moieties such as molecular tags or photosensitizers or the like, via a biotin moiety; (ii) antibodies specific for a target molecule and conjugated to a polymer, such as dextran, which, in turn, is derivatized with moieties such as molecular tags or photosensitizers, either directly by covalent bonds or indirectly via streptavidin-biotin linkages; (iii) antibodies specific for a target molecule and conjugated to a bead, or microbead, or other solid phase support, which, in turn, is derivatized either directly or indirectly with moieties such as molecular tags or photosensitizers, or polymers containing the latter.

“Antigenic determinant” or “epitope” are used interchangeably herein to refer to a site on the surface of a molecule, usually a protein, to which a single antibody molecule binds. Generally, a protein has several or many different antigenic determinants and reacts with antibodies of different specificities. One preferred antigenic determinant is a phosphorylation site of a protein.

As used herein, the terms “aspect” and “embodiment” are used interchangeably.

“Binding compound” shall refer herein to an antibody binding composition, an antibody, a peptide, a peptide or non-peptide ligand for a cell surface receptor, a protein, an oligonucleotide, an oligonucleotide analog, such as a peptide nucleic acid, a lectin, or any other molecular entity that is capable of specifically binding to a target protein or molecule or stable complex formation with an analyte of interest, such as a complex of proteins. In one aspect, a binding compound, which can be represented by the formula below, comprises one or more molecular tags attached to a binding moiety.

“Binding moiety” refers to any molecule to which molecular tags can be directly or indirectly attached that is capable of specifically binding to an analyte. Binding moieties include, but are not limited to, antibodies, antibody binding compositions, peptides, proteins, nucleic acids, and organic molecules having a molecular weight of up to about 1000 daltons and containing atoms selected from the group consisting of hydrogen, carbon, oxygen, nitrogen, sulfur and phosphorus. Preferably, binding moieties are antibodies or antibody binding compositions.

As used herein, “cancer” and “cancerous” refer to or describe the physiological condition organism, including mammals, that is typically characterized by unregulated cell growth. Examples of cancer include, but are not limited to, carcinoma, lymphoma, blastoma, sarcoma and leukemia. More particular examples of such cancers include squamous cell carcinoma, lung cancer, e.g., small-cell lung cancer or non-small cell lung cancer; gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, prostate cancer, vulval cancer, thyroid cancer, hepatic carcinoma and various types of head and neck cancer.

“Chemotherapeutic agent” means a chemical substance, primarily a cytotoxic or cytostatic agent that is used to treat cancer. Chemotherapeutic agents may have specific protein targets on which they act to have an anti-cancer effect (e.g., HER2, HER3). Chemotherapeutic agents shall include such compounds as paclitaxel, as set forth herein.

As used herein, the phrase “cleavable linkage” refers to a chemical linking group that may be cleaved under conditions that do not degrade the structure or affect detection characteristics of a molecular tag connected to a binding moiety with the cleavable linkage.

“Cleavage-inducing moiety” or “cleaving agent” are used interchangeably herein to refer to (1) a group that produces an active species that is capable of cleaving a cleavable linkage, for example, by oxidation, and (2) a chemical compound that can directly cleave a cleavable linkage, for example, by reduction. Preferably, the active species is a chemical species that exhibits short-lived activity so that its cleavage-inducing effects are only in the proximity of the site of its generation. An example of a chemical compound that can directly cleave a cleavable linkage is a reducing agent such as dithiothreitol (DTT), dithioerythritol (DTE), 2-mercaptoethanol or sodium borohydride.

A “cleaving probe,” as used herein, refers to a reagent that comprises a cleavage-inducing moiety that is a group that produces an active species that is capable of cleaving a cleavable linkage and an antibody binding composition, an antibody composition, an antibody, a peptide, a peptide or non-peptide ligand for a cell surface receptor, a protein, such as biotin or streptavidin, an oligonucleotide, an oligonucleotide analog, such as a peptide nucleic acid, a lectin or any other molecular entity that is capable of specifically binding to a target protein or molecule or of having stable complex formation with an analyte of interest, such as a complex of proteins.

“FFPE” or “formalin-fixed, paraffin-embedded” refer to a group of cells or quantity of tissue that are fixed, particularly conventional formalin-fixed, paraffin-embedded samples. Such samples are typically, without limitation, used in an assay for receptor complexes in the form of thin sections, e.g. 3-10 μm thick, of fixed tissue mounted on a microscope slide or equivalent surface. Such samples also typically undergo a conventional re-hydration procedure, and optionally, an antigen retrieval procedure as a part of, or preliminary to, assay measurements. In some embodiments, the sections are about 5 μm thick sections of fixed tissue cut onto positively charged glass sides.

As used herein, “greater than or equal to” (i.e., ≧ or >=) can in certain alternative embodiments mean “greater than” (>). Also, as used herein, “less than or equal to” (i.e., ≦ or <=) can in certain alternative embodiments mean “less than” (<).

“Hazard ratio”, as used herein, refers to a statistical method used to generate an estimate for relative risk. “Hazard ratio” is the ratio between the predicted hazard of one group versus another group. For example, patient populations treated with, versus without, a HER3-acting agent can be assessed for whether or not the HER3-acting agent is effective in increasing the time to distant recurrence of disease. The hazard ratio can then be compared to an independent measure, such as the ratio of activated HER3 to total HER3. At ratios of activated HER3 to total HER3 at which the hazard ratio is less than one, treating with a HER3-acting agent has a greater chance of efficacy. At ratios of activated HER3 to total HER3 at which the hazard ratio is indistinguishable from one, treating with a HER3 acting agent has a lower chance of efficacy.

“HER1,” “Her1,” “Her-1,” “EGFR,” “ErbB1,” and the like are used interchangeably herein to refer to native HER1, and allelic variants thereof, as described, for example, in Cohen et al., 1980, J. Biol. Chem. 255:4834-42, and GenBank Accession No. NM_{—}005228 (see http://www.ncbi.nlm.nih.gov/nuccore/NM_{—}005228). Unless indicated otherwise, the terms “HER1,” “Her1,” “Her-1,” “EGFR,” “ErbB1,” and the like, when used herein, refer to the human protein. The gene encoding HER1 is referred to herein as “erbB1.” As used herein, H1T shall refer to total HER1 expression.

“Her-2”, “ErbB2”, “c-Erb-B2”, “HER2”, “Her2” and “neu” are used interchangeably herein and refer to native Her-2, and allelic variants thereof, as described, for example, in Semba et al., 1985, Proc. Nat. Acad. Sci. USA 82:6497-650 and Yamamoto et al., 1986, Nature 319:230-234 and Genebank accession number X03363. Unless indicated otherwise, the terms “Her-2”, “ErbB2”, “c-Erb-B2”, “HER2” and “Her2” when used herein refer to the human protein. The gene encoding Her2 is referred to herein as “erbB2.” As used herein, H2T shall refer to total Her-2 expression. HER2/HER3 heterodimers may be referred to as H23D or H2/3.

“HER2-acting agent” or “HER2-targeted therapy”, as used herein, refers to a compound that can inhibit a biological activity of HER2 or a HER2 expressing cell or a HER2 positive cancer cell. Such biological activities include, but are not limited to, dimerization, autophosphorylation, phosphorylation of or by another receptor, signal transduction and the like. Biological activities can include, without limitation, cell survival and cell proliferation, and inhibition of such activities by a HER2-acting agent could result in direct or indirect cell killing (e.g., antibody-dependent cellular cytotoxicity (ADCC)), disruption of protein complexes or complex formation, modulation of protein trafficking or enzyme inhibition. Biological activities can also include patient response as set forth in this application. It will be appreciated that, as used herein, HER2-acting agents encompass molecules capable of interfering with, blocking, reducing or modulating the interaction between HER2 and ligands capable of binding to HER2. HER2-acting agents also include bispecific agents that inhibit HER2 and another biological target (e.g., a bispecific antibody or dual kinase inhibitor). Exemplary HER2-targeted agents include, but are not limited to, BIBW 2992, HKI-272, 4D5, pertuzumab, trastuzumab, trastuzumab emtansine, AEE-788, and lapatinib.

“Her-3”, “ErbB3”, “c-Erb-B3”, “HER3”, and “Her3” are used interchangeably herein and refer to native HER3, and allelic variants thereof, as described, for example, in Kraus, M. H., Issing, W., Miki, T., Popescu, N. C., and Aaronson, S. A. (1989) Proc. Natl. Acad. Sci. USA 86, 9193-9197. Plowman, G. D., Whitney, G. S., Neubauer, M. G., Green, J. M., McDonald, V. L., Todaro, G. J., Shoyab, M. (1990) Proc. Natl. Acad. Sci USA 87,4905-4090 and GenBank Accession Nos. NM_{—}001005915 and NM_{—}001982. Unless indicated otherwise, the terms “HER3,” “Her3,” “Her-3,” “ErbB3,” and the like, when used herein, refer to the human protein. The gene encoding HER3 is referred to herein as “erbB3.” As used herein, H3T shall refer to total HER3 expression. HER2/HER3 heterodimers may be referred to as H23D or H2/3.

“HER3-acting agent” or “HER3-targeted therapy”, as used herein, refers to a compound that can inhibit a biological activity of HER3 or a HER3 expressing cell or a HER3 positive cancer cell. Such biological activities include, but are not limited to, dimerization, autophosphorylation, phosphorylation of or by another receptor, signal transduction and the like. Biological activities can include, without limitation, cell survival and cell proliferation, and inhibition of such activities by a HER3-acting agent could result in direct or indirect cell killing (e.g., antibody-dependent cellular cytotoxicity (ADCC)), disruption of protein complexes or complex formation, modulation of protein trafficking or enzyme inhibition. Biological activities can also include patient response as set forth in this application. It will be appreciated that, as used herein, HER3-acting agents encompass molecules capable of interfering with, blocking, reducing or modulating the interaction between HER3 and ligands capable of binding to HER3. HER3-acting agents also include bispecific agents that inhibit HER3 and another biological target (e.g., a bispecific antibody or dual kinase inhibitor). Exemplary HER3-acting agents include, but are not limited to, large molecules (such as antibodies) or small molecules (such as small molecule kinase inhibitors) targeted to HER3, PI3K, Akt, mTOR, ERK1/2, or PYK2. For example, HER3-targeted agents include, but are not limited to, U3-1289/AMG888, MM-121/SAR256212, MM-111, MEHD7945A, AZD-8931, LJM716, Av-203, and pertuzumab (binds HER2 but blocks HER2-HER3 heterodimer formation).

As used herein, “HER3/PI3K complexes” refers to an activated HER3 to which a p85 regulatory subunit (p85) of phosphatidylinositide 3-kinase is bound. HER3 is the principal HER family member that can activate the PI3K/Akt pathway directly. Generally, HER3 is activated upon dimerization with another HER protein (e.g., HER2), which results in phosphorylation of tyrosines in the cytoplasmic domain of the protein. These phosphorylated tyrosine residues act as recruitment sites for p85-PI3K binding, which activates the catalytic subunit of PI3K, p110-PI3KCa, which in turn leads to intracellular signaling via the PI3K/Akt pathway. As used herein, HER3/PI3K complexes may be referred to as “HER3-PI3 kinase.”

A “HER3 positive” or “HER3-activated” cancer, cancer cell, subject or patient, as used herein, refers to a cancer cell, subject, or patient, that has elevated levels of activated HER3.

The phrase “activated HER3 to total HER3 ratio” refers to a measure that describes the amount of activated HER3 molecules divided by the total amount of HER3 in a sample from a subject's tissue (e.g., tumor) according to any single quantitative method available to one skilled in the art.

“High” refers to a measure that is greater than normal, greater than a standard such as a predetermined measure or a subgroup measure or that is relatively greater than another subgroup measure. For example, high total HER3 refers to a measure of HER3 that is greater than a measure of HER3 normally observed in normal/healthy cells or, alternatively, in certain cancer cells. For example, high HER3 means a measure of HER3 that is greater than a normal, average, median HER3 measure in a particular set of samples (healthy or tumor). High HER3 may also refer to a measure that is equal to or greater than a predetermined measure, such as a predetermined cutoff. High HER3 may also refer to a measure of HER3 wherein a high HER3 subgroup has relatively greater levels of HER3 than another subgroup. For example, without limitation, according to the present specification, two distinct patient subgroups can be created by dividing samples around a mathematically determined point, such as, without limitation, a median, thus creating a subgroup whose measure is high (i.e., higher than the median) and another subgroup whose measure is low. HER3 can be measured by any method known to one skilled in the art such as, for example, without limitation, using VeraTag® or using any standard immunohistochemical (IHC) method.

As another example, high activated HER3 refers to a measure of activated HER3 that is greater than a measure of activated HER3 observed in a particular set of biological samples (healthy/normal cells or tumor samples). High activated HER3 may also refer to a measure that is greater than a predetermined measure, such as a predetermined cutoff. High activated HER3 may also refer to a measure of activated HER3 wherein a highly activated HER3 subgroup has a relatively higher level of activated HER3 than another subgroup. Activated HER3 can be measured by methods known in the art such as fluorescence resonance energy transfer (FRET), bioluminescent resonance energy transfer (BRET), proximity ligation assay (PLA), dimer-specific antibodies or VeraTag® (Monogram Biosciences, CA) or any other method that is well known to one skilled in the art.

In some cases, a “high” expression level may comprise a range of expression that is very high and a range of expression that is “moderately high” where moderately high is a level of expression that is greater than normal, but less than “very high.” As another example, high activated HER3 to total HER3 ratio may refer to one or more subgroups of activated HER3 to total HER3 ratios that have measures greater than low ratio subgroups.

“Moderately high” “medium” or “intermediate”, as used herein, refers to a measure that is greater than “low” and less than very “high”. For example, “intermediate” may be used to describe one or more of the subgroups that fall in the middle range of measures of total HER3 and activated HER3 to total HER3 ratios.

“Likely to,” as used herein, refers to an increased probability that an item, object, thing or person will occur. Thus, in one example, a subject that is likely to respond to treatment with a HER3-acting agent, such as cetuximab, has an increased probability of responding to treatment with the HER3-acting agent relative to a reference subject or group of subjects.

“Long,” as used herein, refers to a time measure that is greater than normal, greater than a standard such as a predetermined measure or a subgroup measure that is relatively longer than another subgroup measure. For example, with respect to a patient's longevity, a long time progression refers to time progression that is longer than a normal or average or median time progression observed in subjects having the same type of cancer. Whether a time progression is long or not may be determined according to any method available to one skilled in the art. Long could include, for example, no progression. In one embodiment, “long” refers to a time that is greater than the median time course required for a significant event to occur in a disease.

“Low” is a term that refers to a measure that is less than normal, less than a standard such as a predetermined measure, or a subgroup measure that is relatively less than another subgroup measure. For example, low total HER3 refers to a measure of HER3 that is less than a measure of HER3 normally observed in normal/healthy cells or, alternatively, in certain cancer cells. For example, low HER3 means a measure of HER3 that is less than a normal, average, median HER3 measure in a particular set of samples (healthy or tumor). Low HER3 may also mean a measure that is less than a predetermined measure, such as a predetermined cutoff Low HER3 may also mean a measure wherein a low HER3 subgroup is relatively lower than another subgroup. For example, without limitation, according to the present specification, two distinct patient subgroups can be created by dividing samples around a mathematically determined point, such as, without limitation, a median, thus creating a group whose measure is low (i.e., less than the median) with respect to another group whose measure is high. HER3 can be measured by methods known to one skilled in the art such as, for example, without limitation, using a VeraTag® assay or using any standard immunohistochemical (IHC) method.

As another example, low activated HER3 means a measure of activated HER3 that is less than a normal measure of activated HER3 observed in a particular set of biological samples (healthy/normal cells or tumor samples). Low activated HER3 may also mean a measure that is less than a predetermined measure, such as a predetermined cutoff. Low activated HER3 may also mean a measure wherein a low activated HER3 subgroup is relatively less than another subgroup. HER3-containing dimers (e.g., HER2/HER heterodimers) can be measured by methods known in the art such as Fluorescence resonance energy transfer (FRET), Bioluminescent resonance energy transfer (BRET), proximity ligation assay (PLA), dimer-specific antibodies or VeraTag® or any other method that is well known to one skilled in the art. As another example, low activated HER3 to total HER3 ratio may refer to the one or more subgroups of activated HER3 to total HER3 ratios that have measures less than either intermediate or high ratio subgroups. Low activated HER3 to total HER3 ratios may be determined according to any individual quantitative method available to one skilled in the art. Example ranges for low values of HER3 expression are provided herein.

A “molecular tag,” as used herein, refers to a molecule that can be distinguished from other molecules based on one or more physical, chemical or optical differences among the molecules being separated, including but not limited to, electrophoretic mobility, molecular weight, shape, solubility, pKa, hydrophobicity, charge, charge/mass ratio, polarity or the like. In one aspect, molecular tags in a plurality or set differ in electrophoretic mobility and optical detection characteristics and can be separated by electrophoresis. In another aspect, molecular tags in a plurality or set may differ in molecular weight, shape, solubility, pKa, hydrophobicity, charge, polarity and can be separated by normal phase or reverse phase HPLC, ion exchange HPLC, capillary electrochromatography, mass spectroscopy, gas phase chromatography or like technique. As described herein, a VeraTag® reporter molecule is a type of molecular tag.

“Optimal cutoff as used herein, refers to the value of a predetermined measure on subjects exhibiting certain attributes that allow the best discrimination between two categories of an attribute. For example, finding a value for an optimal cutoff that allows one to best discriminate between two categories (e.g., high H3T expression and low H3T expression). Optimal cutoffs are used to separate the subjects with values lower than or higher than the optimal cutoff to optimize the prediction model, for example, without limitation, to maximize the specificity of the model, maximize the sensitivity of the model, maximize the difference in outcome, or minimize the p-value from hazard ratio or a difference in response.

“Overall survival” or “OS” refers to a time as measured from the start of treatment to death or censor. Censoring may come from a study end or change in treatment. Overall survival can refer to a probability as, for example, a probability when represented in a Kaplan-Meier plot of being alive at a particular time, that time being the time between the start of the treatment to death or censor.

“Photosensitizer” shall mean a light-adsorbing molecule that when activated by light converts molecular oxygen into singlet oxygen.

“RECIST” shall mean an acronym that stands for “Response Evaluation Criteria in Solid Tumours” and is a set of published rules that define when cancer patients improve (“respond”), stay the same (“stable”) or worsen (“progression”) during treatments. Response as defined by RECIST criteria have been published, for example, at Journal of the National Cancer Institute, Vol. 92, No. 3, Feb. 2, 2000 and RECIST criteria may include other similar published definitions and rule sets. One skilled in the art would understand definitions that go with RECIST criteria, as used herein, such as “PR,” “CR,” “SD” and “PD.”

“Relative peak area” or “RPA” are used interchangeably and shall refer to the ratio between the fluorescence measurement of a particular VeraTag® reporter molecule and the fluorescence measurement of a known internal fluorescence standard of known and constant concentration.

“Responsiveness,” to “respond” to treatment, and other forms of this verb, as used herein, refer to the reaction of a subject to treatment with a Her-2-acting agent. As an example, a subject responds to treatment with a Her2-acting agent if growth of a tumor in the subject is retarded about 10%, 20%, 30%, 40%, 50%, 60%, 70%, 80%, 90% or more. In another example, a subject responds to treatment with a Her-2-acting agent if a tumor in the subject shrinks by about 5%, 10%, 20%, 30%, 40%, 50% or more as determined by any appropriate measure, e.g., by mass or volume. In another example, a subject responds to treatment with a Her2-acting agent if the subject experiences a life expectancy extended by about 5%, 10%, 20%, 30%, 40%, 50% or more beyond the life expectancy predicted if no treatment is administered. In another example, a subject responds to treatment with a Her-2-acting agent if the subject has an increased disease-free survival, overall survival or increased time to progression. Several methods may be used to determine if a patient responds to a treatment including the RECIST criteria, as set forth above.

The terms “sample,” “tissue sample,” “patient sample,” “patient cell or tissue sample,” or “specimen” each refer to a collection of similar cells obtained from a tissue of a subject or patient. The source of the tissue sample may be solid tissue as from a fresh, frozen, and/or preserved organ or tissue sample or biopsy or aspirate; blood or any blood constituents; bodily fluids such as cerebral spinal fluid, amniotic fluid, peritoneal fluid or interstitial fluid; or cells from any time in gestation or development of the subject. The tissue sample may contain compounds that are not naturally intermixed with the tissue in nature such as preservatives, anticoagulants, buffers, fixatives, nutrients, antibiotics or the like. Cells may be fixed in a conventional manner (e.g., formalin-fixed, paraffin-embedding (FFPE)).

“Short,” as used herein, refers to a time measure that is shorter than normal, shorter than a standard such as a predetermined measure or a subgroup measure that is relatively shorter than another subgroup measure. For example, with respect to a patient's longevity, a short time progression refers to time progression that is shorter than a normal or average or median time progression observed in subjects having the same type of cancer. Whether a time progression is short or not may be determined according to any method available to one skilled in the art. In one embodiment, “short” refers to a time that is less than the median time course required for a significant event to occur in a disease.

As used herein, “significant event” shall refer to an event in a patient's disease that is clinically important as determined by one skilled in the art. Examples of significant events include, for example, without limitation, primary diagnosis, death, recurrence, the determination that a patient's disease is metastatic, relapse of a patient's disease or the progression of a patient's disease from any one of the above noted stages to another. A significant event may be any clinically important event used to assess overall survival (OS), progression free survival (PFS), disease free survival (DFS), or time to progression (TTP). Some significant events can be determined using the RECIST or other response criteria, as determined by one skilled in the art.

As used herein, the terms “subject” and “patient” are used interchangeably. As used herein, the terms “subject” and “subjects” refer to an animal, preferably a mammal including a non-primate (e.g., a cow, pig, horse, donkey, goat, camel, cat, dog, guinea pig, rat, mouse, sheep) and a primate (e.g., a monkey, such as a cynomolgous monkey, gorilla, chimpanzee and a human).

As used herein, “time course” shall refer to the amount of time between an initial event and a subsequent event. For example, with respect to a patient's cancer, time course may relate to a patient's disease and may be measured by gauging clinically significant events in the course of the disease using, e.g., the RECIST criteria or other response criteria. For example, an intial event may be diagnosis and the subsequent event may be metastasis.

“Time to progression” or “TTP” refers to a time as measured from the start of a treatment to progression of a cancer or censor. Censoring may come from a study end or from a change in treatment. Time to progression can also be represented as a probability as, for example, in a Kaplan-Meier plot where time to progression may represent the probability of being progression free over a particular time (time between the start of the treatment to progression or censor).

“Treat,” “treatment,” and other forms of this word refer to the administration of an agent to impede growth of a cancer, to cause a cancer to shrink by weight or volume, to extend the expected survival time of the subject and or time to progression of the tumor or the like.

“Unlikely to” refers to a decreased probability that an event, item, object, thing or person will occur with respect to a reference. Thus, a subject that is unlikely to respond to treatment with a HER3-acting agent has a decreased probability of responding to treatment with a HER3-acting agent relative to a reference subject or group of subjects.

“VeraTag®” and “VeraTag® assay” are used interchangeably herein and refer to single and multiplexed immunoassays, both single- and multi-label format, and the materials, methods and techniques for performing and utilizing such assays, including but not limited to reagents, analytical procedures and software related to those assays (Monogram Biocsiences, CA). Labels in the context of a VeraTag® assay are detectable moieties that are referred to as VeraTag® reporter molecules. Such assays are disclosed in this application as well as in U.S. Pat. No. 7,648,828 and U.S. Patent Application Nos. 2010/0143927; 2010/0233732; and 2010/0210034, which are incorporated by reference herein in their entireties.

As used herein, “VeraTag® reporter molecule” or “vTag,” are used to refer to a detectable moiety that is attached to an antibody used in a VeraTag® assay (Monogram Biosciences, CA).

### Methods Involving Analysis of HER3

Aspects and embodiments of the invention provide systems and methods for facilitating diagnosis, prognosis and treatment of cancer based on detection of HER3 activation. In certain embodiments, the methods involve determining whether a subject with a cancer is likely to respond to treatment with a HER3-acting agent and/or for predicting a time course of disease and/or a probability of a significant event in the time course of disease in a subject with a cancer. In certain embodiments, the method comprises detecting activated HER3, alone or in a combination with other biomarkers associated with responsiveness to treatment with a HER3-acting agent as described herein, and determining whether the subject is likely to respond to treatment with the HER3-acting agent alone or in combination with another agent (e.g., HER2-acting agent). In certain embodiments, the methods comprise measuring activated HER3 as a biomarker or in a combination of biomarkers and predicting a time course associated with progression of disease or a probability of a significant event in the time course of disease in a subject with cancer. In some embodiments, the methods comprise measuring activated HER3 and determining an appropriate treatment for a subject (e.g., HER3-targeted agent).

In one aspect, the invention provides methods for measuring the amount of activated HER3 in a tumor, comprising: (a) providing a sample from a tumor; (b) measuring the amount of total HER3 in the sample and the amount of at least one of HER2-HER3 heterodimer, phosphorylated HER3, or HER3/PI3K complex in the sample; and (c) determining the ratio of at least one of HER2-HER3 heterodimer, phosphorylated HER3, or HER3/PI3K complex to total HER3 protein.

In another aspect, the invention provides methodsfor measuring the amount of activated HER3 in a tumor, comprising (a) measuring in a tumor sample the amount of total HER3 and the amount of at least one of HER2-HER3 heterodimer, phosphorylated HER3, or HER3/PI3K complex in the sample; (b) determining the ratio of at least one of HER2-HER3 heterodimer, phosphorylated HER3, or HER3/PI3K complex to total HER3 protein; and (c) indicating that the tumor has a high amount of activated HER3 if (i) the amount of total HER3 in the sample is above the median amount of total HER3 of a reference population and (ii) the ratio of at least one of HER2-HER3 heterodimer to HER3 total, phosphorylated HER3 to total HER3 in the sample, or HER3/PI3K complex is above the median ratio of at least one of HER2-HER3 heterodimer to HER3 total, phosphorylated HER3 to total HER3, or or HER3/PI3K complex to total HER3 in the reference population.

In another aspect, the invention provides methods of treating a subject with cancer comprising: (a) determining whether the subject's cancer has high amounts of activated HER3 by (i) measuring the amount of total HER3 protein and at least one of HER2-HER3 heterodimer, phosphorylated HER3, or HER3/PI3K complex in a tumor sample from the subject, and (ii) determining if the tumor sample comprises elevated amounts of total HER3 protein and at least one of HER2-HER3 heterodimer, phosphorylated HER3, or HER3/PI3K complex; and (b) administering a HER3-targeted therapy to the subject.

In another aspect, the present invention provides methods of treating a subject with cancer comprising: (a) measuring in a tumor sample from the subject the amount of total HER3 protein and at least one of HER2-HER3 heterodimer, phosphorylated HER3, or HER3/PI3K complex; (b) determining the ratio of the amount of at least one of HER2-HER3 heterodimer, phosphorylated HER3, or HER3/PI3K complex to total HER3; (c) determining if a subject has a cancer characterized as having a high level of activated HER3, wherein a high level of activated HER3 comprises (i) the amount of total HER3 in the sample being above the median amount of total HER3 of a reference population of subjects having the same type of cancer as the subject and (ii) the ratio of at least one of the amount of HER2-HER3 homodimer to the amount of HER3 total, the amount of phosphorylated HER1 to the amount of total HER3, or the amount of HER3-PI3K complex to the amount of HER3 total in the sample being above the median ratio of at least one of the amount of HER2-HER3 homodimer to the amount of HER3 total, the amount of phosphorylated HER3 to the amount of total HER3, or the amount of HER3-PI3K complex to the amount of HER3 total, in the reference population of subjects having the same type of cancer as the subject; and (d) administering a HER3-targeted therapy to the subject if the subject has a cancer characterized as having a high level of activated HER3.

In another aspect, the invention provides methods for predicting responsiveness of a subject with cancer to a HER3 acting agent comprising: (a) measuring the amount of total HER3 protein and at least one of HER2-HER3 heterodimer, phosphorylated HER3, or HER3/PI3K complex in a tumor sample from the subject; (b) indicating that the subject is more likely to respond to the HER3 acting agent if (i) the amount of total HER3 in the sample is above the median amount of total HER3 of a reference population and (ii) the ratio of at least one of HER2-HER3 heterodimer to HER3 total, phosphorylated HER3 to total HER3 in the sample, or HER3/PI3K complex is above the median ratio of at least one of HER2-HER3 heterodimer to HER3 total, phosphorylated HER3 to total HER3, or HER3/PI3K complex to total HER3 in the reference population. In certain aspects of the invention, responsiveness to a HER3-acting agent comprises a longer disease time course between diagnosis or initiation of treatment and the occurrence of a significant event while the subject is being treated with a HER3-acting agent. In some aspects of the invention, the significant event comprises at least one of progression of the cancer from one stage to a more advanced stage, progression to metastatic disease, relapse, surgery, or death.

In another aspect, the present invention provides methods for predicting responsiveness of a subject with cancer to a HER3 acting agent comprising: (a) measuring in a tumor sample from the subject the amount of total HER3 protein and at least one of HER2-HER3 heterodimer, phosphorylated HER3, or HER3/PI3K complex; (b) determining the ratio of the amount of at least one of HER2-HER3 heterodimer, phosphorylated HER3, or HER3/PI3K complex to total HER3; (c) determining if a subject has a cancer characterized as having a high level of activated HER3, wherein a high level of activated HER3 comprises (i) the amount of total HER3 in the sample being above the median amount of total HER3 of a reference population of subjects having the same type of cancer as the subject and (ii) the ratio of at least one of the amount of HER2-HER3 homodimer to the amount of HER3 total, the amount of phosphorylated HER1 to the amount of total HER3, or the amount of HER3-PI3K complex to the amount of HER3 total in the sample being above the median ratio of at least one of the amount of HER2-HER3 homodimer to the amount of HER3 total, the amount of phosphorylated HER3 to the amount of total HER3, or the amount of HER3-PI3K complex to the amount of HER3 total, in the reference population of subjects having the same type of cancer as the subject; and (d) indicating that the subject is more likely to respond to the HER3 acting agent if the subject's cancer is characterized as having a high level of activated HER3. In some aspects of the invention, the methods may further comprise indicating that the subject is more likely to respond to a co-therapy of a HER2-targeted agent and a HER3-targeted agent if the subject has a HER2 positive cancer. In certain aspects of the invention, responsiveness to a HER3-acting agent comprises a longer disease time course between diagnosis or initiation of treatment and the occurrence of a significant event while the subject is being treated with a HER3-acting agent. In some aspects of the invention, the significant event comprises at least one of progression of the cancer from one stage to a more advanced stage, progression to metastatic disease, relapse, surgery, or death.

Additional aspects and embodiments of the invention relating to each of the above embodiments are described below.

In some embodiments of the invention, the methods may further comprise indicating that the tumor/cancer has a high amount of activated HER3 if (i) the amount of total HER3 in the sample is above the median amount of total HER3 of a reference population and (ii) the ratio of at least one of HER2-HER3 heterodimer to HER3 total, phosphorylated HER3 to total HER3 in the sample, or HER3/PI3K complex is above the median ratio of at least one of HER2-HER3 heterodimer to HER3 total, phosphorylated HER3 to total HER3, or or HER3/PI3K complex to total HER3 in the reference population. In certain embodiments of the invention, the amount of activated HER3 in the cancer/tumor is detected by determining the amount of at least two HER3 entities selected from the group consisting of HER2-HER3 heterodimer, phosphorylated HER3, and HER3/PI3K complex that is present in the biological sample or tumor sample.

In some embodiments of the invention, the amount of phosphorylated HER3 in the biological sample or tumor sample is detected by using a HER3 phosphospecific or a HER3 pan-phospo antibody. In certain embodiments, the amount of phosphorylated HER3 in the tumor is detected by using a phosphospecific antibody that binds HER3 protein that is phosphorylated at the tyrosine residue at position 1289 of HER3.

In some embodiments of the invention, the cancer/tumor that the described methods relate to may comprise at least one of colorectal cancer, gastric cancer, breast cancer, melanoma, ovarian cancer, head and neck cancer, lung cancer (e.g., non-small cell lung cancer), brain cancer, endometrial cancer, pancreatic cancer, prostate cancer, or cervical cancer. In one embodiment, the cancer is breast cancer. In some embodiments, the tumor may comprise breast cancer. In some embodiments, the cancer is metastatic or recurrent. In some embodiments, the cancer is early stage cancer. Alternatively, any cancer that may be sensitive to a HER3-acting agent may be analyzed or monitored.

In some embodiments, the invention relates to use of HER3-targeted agents. A HER3-targeted agent can be any such agent known to one of skill in the art. In certain embodiments the HER3-targeted agent comprises a HER3-specific antibody, including bispecific antibodies, or a protein kinase inhibitor. In certain embodiments the HER3-targeted agent is at least one of U3-1289/AMG888, MM-121/SAR256212, MM-111, MEHD7945A, AZD-8931, LJM716, Av-203, or pertuzumab (binds HER2 but blocks HER2-HER3 heterodimer formation. Also, other HER3-targeted agents may be evaluated using the methods described herein.

In certain embodiments of each of the methods of the invention, the samples may be analyzed for at least one other biomarker. For example, in some embodiments, the at least one other biomarker may also be measured. For example in some embodiments, the other biomarker may comprise total HER2. In other embodiments, the at least one other biomarker is selected from the group consisting of PTEN, MET, STK11, BRAF, KRAS, NRAS, MAP3K1, AKT1, IGF1R, PI3KR1, CCND1, STATS, FGFR-1, and FGFR-4.

In certain embodiments, the method comprises detecting in a biological sample from the subject's cancer the amount of total HER3 and/or activated HER3 wherein if the amount of total HER3 and activated HER3 is high, then the patient is likely to respond to the HER3-acting agent and/or the patient has a long time course.

Thus, in certain embodiments, the invention comprises methods to correlate the relative levels of the amount of activated HER3 in a biological sample from a subject with a prognosis for the likelihood that the subject will respond to treatment with a HER3-acting agent comprising: (a) detecting in a biological sample from the subject's cancer the amount of activated HER3; and (b) correlating the amount of activated HER3 to a prognosis for the likelihood that the subject will respond to treatment with a HER3-acting agent.

In certain embodiments, if the amount of the activated HER3 is equal to or above a first cutoff, the subject's prognosis is to be likely to respond to the HER3-acting agent. Alternatively, if the amount of the activated HER3 is lower than the first cutoff level, the subject's prognosis is to be unlikely to respond to the HER3-acting agent. In some embodiments, the first cutoff comprises the median level of activated Her3 in a reference population of subject with the same cancer. In certain embodiments, the median level of activated HER3 in the reference population comprises a predetermined measure. In some embodiments, activated HER3 is the measure of HER3 total plus the ratio of at least one of HER2/HER3 heterodimers, phosphorylated HER3 or HER3/PI3K complex to HER3 total.

Also, in certain embodiments, a predetermined measure is created by dividing a plurality of subject samples into at least two subgroups, wherein the first subgroup comprises samples having total HER3 molecules at a low level in the biological sample, wherein the low level comprises having an amount of total HER3 molecules equal to or below a threshold level (cutoff); and wherein the second subgroup comprises samples having total HER3 molecules at a high level, wherein the high level comprises having an amount of activated HER3 molecules above the threshold level (cutoff). In other embodiments of the methods, a predetermined measure is generated by dividing the high level total HER3 subgroup into at least two subgroups based on the level of activated HER3 as determined by detecting the amount of HER2/HER3 heterodimer, phosphorylated HER3, or HER3/PI3K complexes. In some embodiments, the cutoff is the median in a reference population.

In certain embodiments, the method comprises detecting in a biological sample from the subject's cancer the amount of total HER3 and/or activated HER3, wherein if the amount of total HER3 and/or activated HER3 is high, then the patient is likely to respond to the HER3-acting agent and/or the patient has a long time course. In some embodiments, activated HER3 is measured.

In other embodiments, the invention is drawn to a method for predicting a time course of disease in a subject with a cancer having elevated levels of activated HER3. In other embodiments, the invention is drawn to a method for predicting the probability of a significant event in a subject with a HER3 positive cancer. For example, some embodiments of the invention comprise methods for predicting whether a subject with a cancer and being treated with a HER3-acting agent is likely to have a significant event comprising the steps of: (a) detecting in a biological sample from the subject's cancer the amount of total HER3 and/or activated HER3; and (b) correlating the amount of total HER3 and/or activated HER3 to the likelihood that the subject will have a significant event. In some embodiments, the significant event is a reduced time between diagnosis with the cancer and at least one of progression of the cancer from one stage to a more advanced stage, progression to metastatic disease, relapse, surgery or death. Also in certain embodiments, the method may further comprise predicting a time course during which the significant event can occur. In certain embodiments, a time course is measured by determining the time between significant events in the course of a patient's disease, wherein the measurement is predictive of whether a patient has a long time course. In one aspect and embodiment, the significant event is the progression from primary diagnosis to death. In another aspect and embodiment, the significant event is the progression from primary diagnosis to metastatic disease. In yet another aspect and embodiment, the significant event is the progression from primary diagnosis to relapse. In another aspect and embodiment, the significant event is the progression from metastatic disease to death. In another aspect and embodiment, the significant event is the progression from metastatic disease to relapse. In another aspect and embodiment, the significant event is the progression from relapse to death. In certain embodiments, the time course is measured with respect to overall survival rate, time to progression and/or using the RECIST or other response criteria.

In another embodiment, the invention comprises a method for determining whether a subject with a cancer having elevated levels of activated HER3 is likely to respond to treatment with a HER3-acting agent and/or have a long disease time course.

In certain embodiments, the subject may be administered a combination therapy that includes a HER3-acting agent. The combination therapy can include the HER3-acting agent in combination with one or more of any chemotherapeutic agent known to one of skill in the art without limitation. Preferably, the chemotherapeutic agent has a different mechanism of action from the HER3-acting agent. For example, the chemotherapeutic agent can be an anti-metabolite (e.g., 5-flourouricil (5-FU), methotrexate (MTX), fludarabine, etc.), an antimicrotubule agent (e.g., vincristine; vinblastine; taxanes such as paclitaxel and docetaxel; etc.), an alkylating agent (e.g., cyclophosphamide, melphalan, bischloroethylnitrosurea, etc.), platinum agents (e.g., cisplatin, carboplatin, oxaliplatin, JM-216, CI-973, etc.), anthracyclines (e.g., doxorubicin, daunorubicin, etc.), antibiotic agents (e.g., mitomycin-C, actinomycin D, etc.), topoisomerase inhibitors (e.g., etoposide, camptothecins, etc.) or other any other chemotherapeutic agents known to one skilled in the art.

Particular examples of chemotherapeutic agents that can be used in the various embodiments of the invention, including pharmaceutical compositions, dosage forms, and kits of the invention, include, without limitation, cytarabine, melphalan, topotecan, fludarabine, etoposide, idarubicin, daunorubicin, mitoxantrone, cisplatin paclitaxel, and cyclophosphamide.

Other chemotherapeutic agents that may be used include abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate.

In another aspect, the invention is drawn to a method for determining whether a subject with a HER3 activated cancer is unlikely to respond to treatment with at least one chemotherapeutic agent in addition to a HER3-acting agent and/or the patient is likely to have a short time course. In certain embodiments, the method comprises measuring in a biological sample from the subject's cancer an amount of HER3 and/or activated HER3, wherein if the level of HER3 and/or activated HER3 is high or very high, then the patient is unlikely to respond to at least one chemotherapeutic agent in addition to a HER3 acting agent.

In another aspect, the invention is drawn to a method for determining whether a subject with a HER3 positive cancer is likely to respond to treatment with at least one chemotherapeutic agent in addition to a HER3-acting agent. In certain embodiments, the method comprises measuring in a biological sample from the subject's cancer an amount of activated HER3, wherein if the level of activated HER3 is high, then the patient is likely to respond to at least one chemotherapeutic agent in addition to the HER3-acting agent. In certain embodiments, the biological sample comprises FFPE tissues. In certain embodiments, the cancer is metastatic or recurrent. In some embodiments, any cancer that may be sensitive to a HER3-acting agent may be monitored. The HER3-acting agent may be any HER3-acting agent. In certain embodiments, the HER3-acting agent is one of the agents described herein. Alternatively, other additional chemotherapeutic agents as known in the art may be evaluated. In certain embodiments, likeliness to respond or time course is measured with respect to overall survival rate, time to progression and/or using the RECIST criteria.

A HER2-targeted agent can be any such agent known to one of skill in the art. In certain embodiments the HER2-targeted agent may be at least one of BIBW 2992, HKI-272, 4D5, pertuzumab, trastuzumab, trastuzumab emtansine, AEE-788, or lapatinib. In certain embodiments, likeliness to respond is measured with respect to overall survival rate, time to progression and/or using the RECIST or other response criteria.

In some embodiments, the invention may further comprise measuring the amount of total HER2 protein in the biological sample and determining if the subject has a HER2 positive cancer or a HER2 negative cancer. In some embodiments of the invention, the HER2 negative cancers and HER2 positive cancers are characterized by immunohistochemical or in situ hybridization analysis (e.g., at a centralized testing laboratory). In some embodiments, the invention may further comprise administering a co-therapy comprising a HER2-targeted therapy and a HER3-targeted therapy if the subject has a HER2 positive cancer. In some embodiments of the invention, the methods may further comprise indicating that the subject is more likely to respond to a co-therapy of a HER2-targeted agent and a HER3-targeted agent if the subject has a HER2 positive cancer.

In certain embodiments, the invention may further comprise determining if the amount of total HER2 protein is below a first cutoff comprising a level of total HER2 protein corresponding to the bottom 5^{th} percentile of total HER2 protein expression in a reference population of HER2 positive cancers (i.e., HERmark® HER2 negative), if the amount of total HER2 protein is above a second cutoff comprising a level of total HER2 protein corresponding to a top 95^{th} percentile of total HER2 protein expression in a reference population of HER2 negative cancers (i.e., HERmark® HER2 positive), or whether the amount of total HER2 protein is above the first cutoff but below the second cutoff (i.e., HERmark® HER2 positive). In certain embodiments, the invention may further comprise administering a co-therapy comprising a HER2-targeted therapy and a HER3-targeted therapy if the amount of total HER2 protein in the tumor sample is above the first cutoff. In certain embodiments, the invention may further comprise indicating that the subject is more likely to respond to a co-therapy of a HER2-targeted agent and a HER3-targeted agent if the amount of total HER2 protein in the biological sample is above the first cutoff.

As discussed herein, in certain embodiments, the amount of activated HER3 is measured using an assay capable of measuring and/or quantifying an amount of protein-protein interactions in a sample. Any method known to one of skill in the art to be useful for directly measuring the amount of total HER3 expression and/or activated HER3 in a sample may be used. For example, any quantitative assay that determines the amount of HER3 expression can be used to determine how much signal is generated by a cell or cancer as representative of HER3 expression or activation. Such methods may include, but not necessarily be limited to, a VeraTag® assay, FRET, BRET, Biomolecular Fluoresence Complementation and Proximity Ligation Assay. In some embodiments, the signal generated in the VeraTag® assay may be compared to the signal generated from a different assay to determine a correspondence between the two assays.

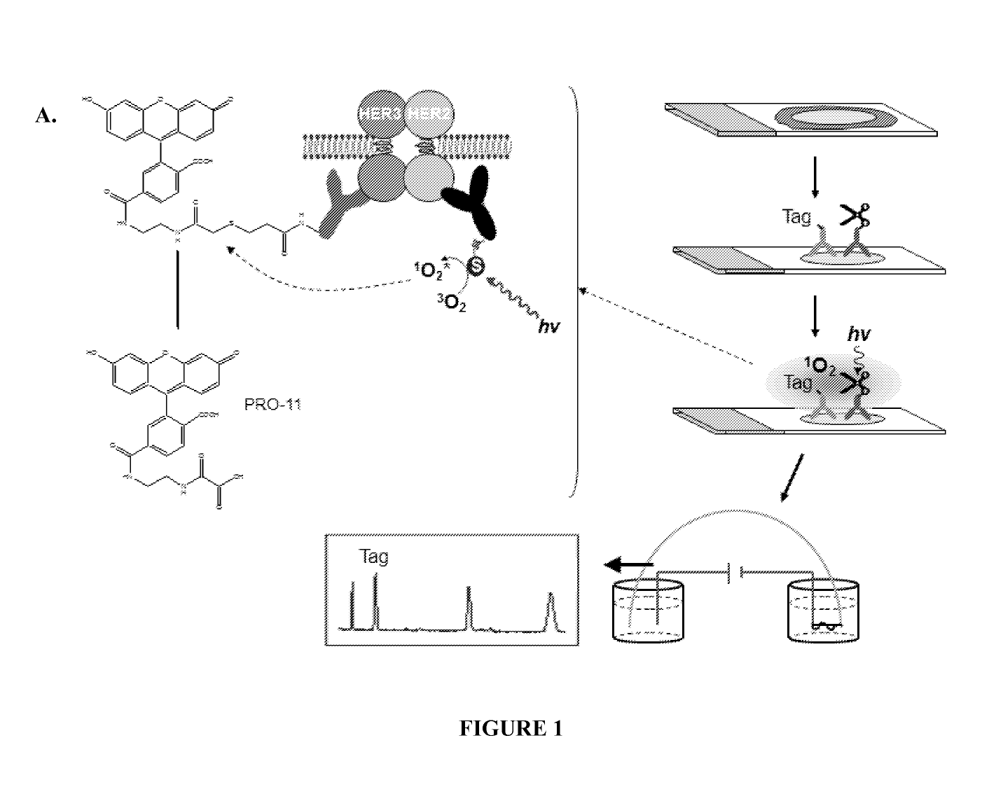

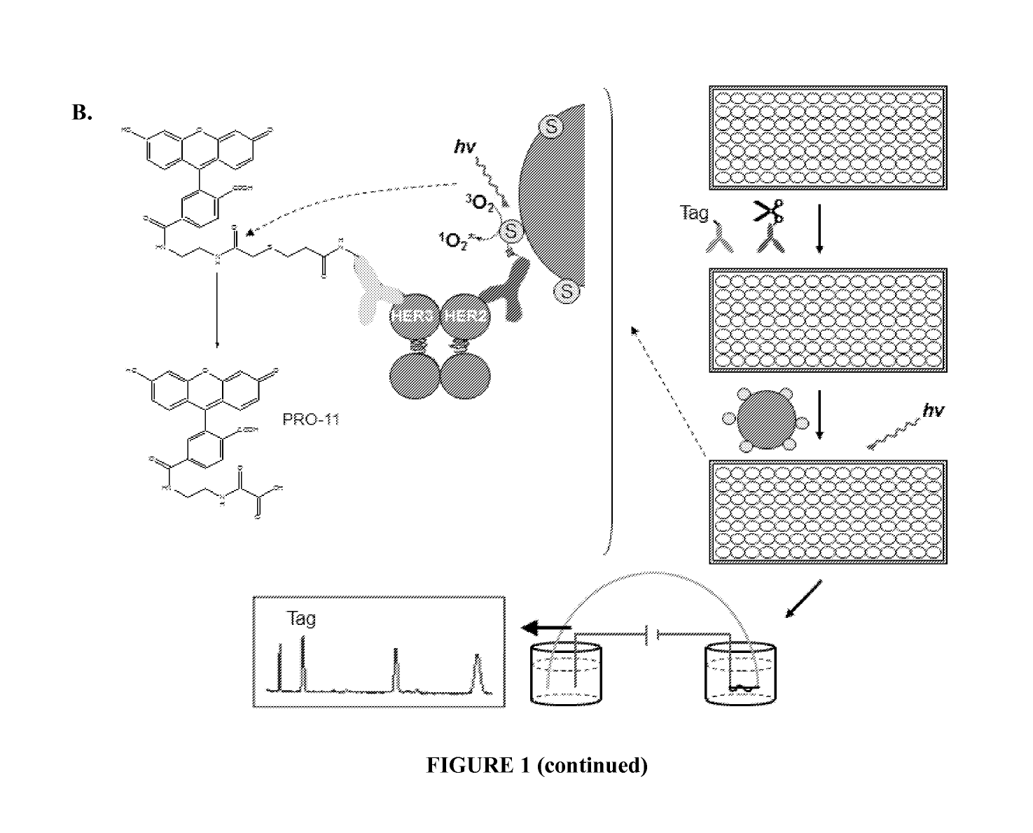

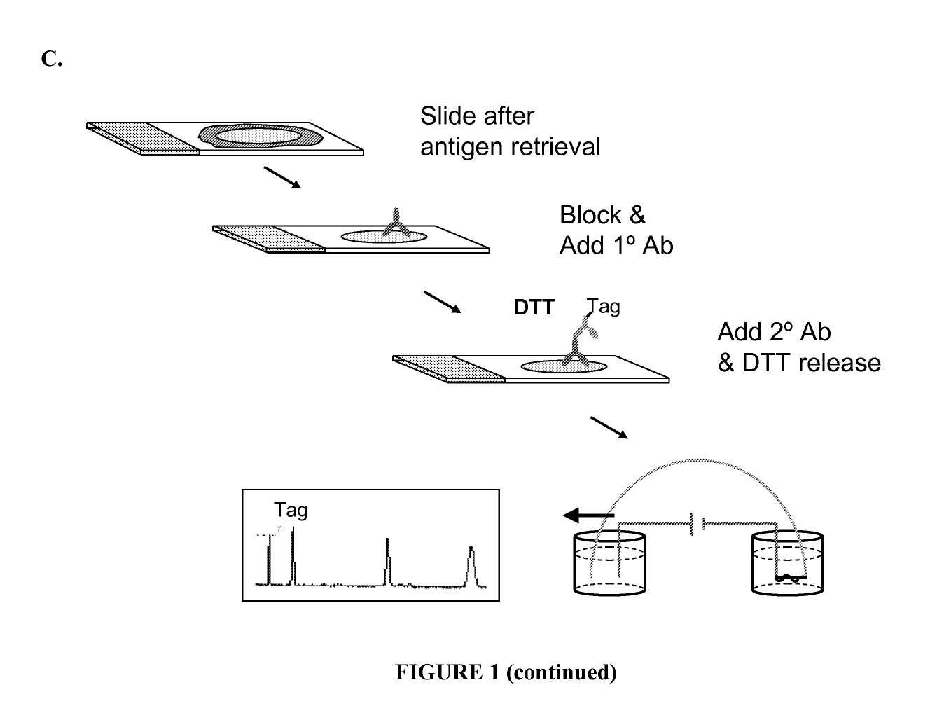

In certain embodiments, the amounts are determined by contacting a biological sample from a subject with cancer with a binding compound having a molecular tag attached thereto by a cleavable linkage and a cleaving probe having a cleavage inducing-moiety and detecting whether and what molecular tag is released. FIG. 1 provides schematic diagrams of various VeraTag® assay formats according to embodiments of the invention. Tissue sections are fixed and then allowed to bind to a first antibody having a cleavage-inducing agent and a second antibody linked to a detectable moiety (e.g., VeraTag® reporter molecule). For example, as shown in FIG. 1A, a first antibody is conjugated to biotin, and a second antibody is conjugated to a VeraTag® reporter molecule. Streptavidin-functionalized sensitizer dye may then be bound to the biotin-conjugated antibody. Photo-induction of the cleavage-inducing agent may be performed to cause the release of singlet oxygen, which induces cleavage of the VeraTag® reporter molecule into the solution. The solution is then collected and analyzed by capillary electrophoresis. In some embodiments, the assay may be performed in microplate format as shown in FIG. 1B. For example, samples are added to the wells of the microplate and then the antibodies are added: a first antibody specific for one target protein is conjugated to biotin, and a second antibody specific for a second target protein is conjugated to a VeraTag® reporter molecule. Beads coated with a streptavidin-functionalized sensitizer dye may be used to capture the biotin-conjugated antibody Photo-induction of the cleavage-inducing agent may be performed to cause the release of singlet oxygen, which induces cleavage of the VₑᵣₐT_{ag}® reporter molecule into the solution. The solution is then collected from each well and analyzed by capillary electrophoresis. In some embodiments, cleavage of the VeraTag® reporter molecule may be caused by DTT instead of photosensitization induced cleavage as shown in FIG. 1C.

Examples of detection assays are described herein as well as in commonly owned U.S. Patent Application Publication No. 2009/0191559, which is incorporated by reference in its entirety herein (describing detection of total HER2 and HER2 homodimers). A similar strategy can be used to measure other biomarkers such as HER1, HER3, cMET, p-95 and the like. See, e.g., U.S. Pat. No. 7,648,828 and U.S. Patent Application Nos. 2010/0143927; 2010/0233732; and 2010/0210034.

In certain embodiments, one of more VeraTag® assay formats may be used to measure HER biomarkers levels. For example, when measuring total HER3 levels, a primary antibody that binds to the extracellular domain of HER3 may used. A secondary antibody with a VeraTag® reporter molecule attached thereto may then be used to specifically bind to the primary antibody. In certain embodiments of this assay format, the VeraTag® reporter molecule may be cleaved with a reducing agent (e.g., DTT) as shown in FIG. 1C.

When measuring amounts of total HER2, total HER3, and phosphorylated HER3, two primary antibodies may be used, both primary antibodies specific for the target protein. One primary antibody may have a methylene blue dye attached and bind to the intracellular domain HER2 or HER3. The second primary antibody is conjugated to a VeraTag® reporter molecule and may also bind the intracellular domain of HER2 or HER3. Upon photosensitization, the methylene blue dye releases singlet oxygen which causes cleavage of the VeraTag® reporter molecule when the molecules are in close proximity. The two primary antibodies may be identical or may bind to two different epitopes of the HER molecule. When measuring HER3 phosphorylation, the VeraTag® reporter molecule-conjugated antibody may bind to a phosphorylated epitope (e.g., amino acid position 1289).

For example, in some embodiments of the invention, measuring the amount of total HER3 protein in a tumor sample or biological sample from the subject may comprise the steps of: a) contacting the tumor sample or biological sample with a HER3 antibody composition; b) contacting the HER3 antibody composition with a tagged binding composition, wherein the tagged binding composition comprises a molecular tag attached thereto via a cleavable linkage, and wherein the tagged binding composition specifically binds to the HER3 antibody composition; c) cleaving the cleavable linker of the tagged binding composition, thereby releasing the molecular tag; and d) quantitating the released molecular tag to determine the amount of HER3 protein in the tumor sample or biological sample.

Also, in some embodiments of the invention, measuring the amount of total HER3 protein in a tumor sample or biological sample from the subject may comprise the steps of: a) contacting the tumor sample or biological sample with a first HER3 antibody composition that specifically binds to HER3 protein at a first binding site, wherein the first HER3 binding composition comprises a molecular tag attached thereto via a cleavable linkage; b) contacting the tumor sample with a cleaving probe that specifically binds to HER3 protein at a second binding site, wherein the cleaving probe cleaves the cleavable linkage of the HER3 antibody composition when within an effective proximity thereto; c) cleaving the cleavable linker of the HER3 antibody composition, thereby releasing the molecular tag; and d) quantitating the released molecular tag to determine the amount of HER3 protein in the tumor sample or biological sample.

When measuring HER2-HER3 heterodimers and HER3/PI3K complexes, the assay format generally uses two primary antibodies that bind each of the proteins being detected and two secondary antibodies that specifically bind to one or the other primary antibodies. The primary antibodies that bind to HER2 and HER3 can bind specifically to either the extracellular domain or the intracellular domain. In some embodiments of the invention, the primary antibody that binds HER3 binds specifically to the intracellular domain when the HER3/PI3K complex is being detected. For example, for detection of HER2/HER3 heterodimers, a HER2 primary antibody can be used in conjunction with a secondary antibody conjugated to a VeraTag® reporter molecule, and a HER3 antibody can be used in conjunction with a secondary antibody that is conjugated to a photosensitizer (e.g., methylene blue). Alternatively, an alternate configuration, the VeraTag® reporter molecule could be conjugated to the secondary antibody that binds the HER3 antibody and the photosensitizer could be conjugated to the secondary antibody that binds the HER2 antibody. Upon photosensitization, if the secondary antibodies are located in close proximity to one another (e.g., if a HER2/HER3 heterodimer has formed), the VeraTag® reporter molecule may be cleaved. Subsequently, the cleaved reporter molecules may then be separated, for example, by electrophoresis. The same assay format can be used for detection of HER3/PI3K complexes using primary antibodies that bind to HER3 and PI3K. In addition, modified formats may be used for detection of HER2/HER3 heterodimers and HER3/PI3K complexes, as described elsewhere herein.

In other assay formats for detection of HER2-HER3 heterodimers and HER3/PI3K complexes, the molecular tag (e.g., VₑᵣₐT_{ag}® reporter molecule) and the cleaving probe having a cleavage inducing-moiety (e.g., photosensitizer), are conjugated directly to the primary antibodies and no secondary antibodies are used. The primary antibodies that bind to HER2 and HER3 can bind specifically to either the extracellular domain or the intracellular domain. In some embodiments of the invention, the primary antibody that binds HER3 binds specifically to the intracellular domain when the HER3/PI3K complex is being detected. For example, in some embodiments of the invention, measuring the amount of HER2-HER3 heterodimer or HER3/PI3K complex in a tumor sample or biological sample from the subject may comprise the steps of: a) contacting the tumor sample or biological sample with an antibody composition comprising a molecular tag attached thereto via a cleavable linkage; b) contacting the tumor sample or biological sample with a cleaving probe, wherein the cleaving probe cleaves the cleavable linkage of the antibody composition when within an effective proximity thereto; c) cleaving the cleavable linker of the antibody composition, thereby releasing the molecular tag; and d) quantitating the released molecular tag to determine the amount of HER2-HER3 heterodimer or HER3/PI3K complex in the tumor sample or biological sample, wherein for measurement of HER2-HER3 heterodimer, the antibody composition binds specifically to HER3 and the cleaving probe binds specifically to HER2, or the antibody composition binds specifically to HER2 and the cleaving probe binds specifically to HER3, and wherein for measurement of HER3/PI3K complex, the antibody composition binds specifically to HER3 and the cleaving probe binds specifically to PI3K, or the antibody composition binds specifically to PI3K and the cleaving probe binds specifically to HER3.

Also, in some embodiments of the invention, measuring the amount of HER2-HER3 heterodimer in a tumor sample or biological sample from the subject may comprise the steps of: a) contacting the tumor sample or biological sample with a HER2 antibody composition; b) contacting the tumor sample or biological sample with a HER3 antibody composition; c) contacting the tumor sample or biological sample with a first binding composition that binds to either the HER2 antibody composition or the HER3 antibody composition, wherein the first binding composition comprises a molecular tag attached thereto via a cleavable linkage; d) contacting the tumor sample or biological sample with a cleaving probe, wherein the cleaving probe cleaves the cleavable linkage of the binding composition when within an effective proximity thereto; e) cleaving the cleavable linker of the antibody composition, thereby releasing the molecular tag; and f) quantitating the released molecular tag to determine the amount of HER2-HER3 heterodimer in the tumor sample or biological sample, wherein the cleaving probe binds specifically to HER2 if the antibody binding composition binds specifically to HER3, or the cleaving probe binds specifically to HER3 if the antibody binding composition binds specifically to HER2.

In addition, in other embodiments of the invention, measuring the amount of HER3/PI3K complex in a tumor sample or biological sample from the subject may comprise the steps of: a) contacting the tumor sample or biological sample with a HER3 antibody composition and a PI3K antibody binding composition, wherein one antibody composition comprises a molecular tag attached thereto via a cleavable linkage and the other antibody composition comprises a cleaving probe that cleaves the cleavable linkage of the binding composition when within an effective proximity thereto; b) cleaving the cleavable linker to release the molecular tag; and c) quantitating the released molecular tag to determine the amount of HER3/PI3K complex in the tumor sample or biological sample.

Also, in some embodiments of the invention, measuring the amount of phosphorylated HER3 in a tumor sample or biological sample from the subject may comprise the steps of: a) contacting the tumor sample or biological sample with a first HER3 antibody composition that specifically binds to HER3 protein at a first binding site, wherein the first HER3 antibody composition comprises a molecular tag attached thereto via a cleavable linkage; b) contacting the tumor sample or biological sample with a second HER3 antibody composition that specifically binds to HER3 protein at a second binding site, wherein the second HER3 antibody composition comprises a cleavage-inducing moiety that cleaves the cleavable linkage of the HER3 antibody composition when within an effective proximity thereto and wherein the second binding site comprises a HER3 phosphorylation site; c) cleaving the cleavable linker of the first HER3 antibody composition, thereby releasing the molecular tag; and d) quantitating the released molecular tag to determine the amount of phosphorylated HER3 in the tumor sample or biological sample.

In some embodiments, samples suitable for use in the systems and methods of the invention may come from a wide variety of sources, including cell cultures, animal tissues, patient biopsies or the like. Preferably, samples are human patient samples. Samples are prepared for analysis of biomarkers using conventional techniques, which may depend on the source from which a sample is taken. For biopsies and medical specimens, guidance is provided in the following references: Bancroft JD & Stevens A, eds. 1977, Theory and Practice of Histological Techniques, Churchill Livingstone, Edinburgh; Pearse, 1980, Histochemistry. Theory and applied. 4^{th} ed., Churchill Livingstone, Edinburgh. In some embodiments, samples comprise FFPE samples.

Examples of patient tissue samples that may be used include, but are not limited to, colorectal cancer, gastric cancer, breast cancer, melanoma, ovarian cancer, head and neck cancer, lung cancer (e.g., non-small cell lung cancer), brain cancer, endometrial cancer, pancreatic cancer, prostate cancer, or cervical cancer. The tissue sample can be obtained by a variety of procedures including surgical excision, aspiration or biopsy. The tissue may be fresh or frozen. In one embodiment, assays of the invention are carried out on tissue samples that have been fixed and embedded in paraffin and a step of deparaffination is then carried out. A tissue sample may be fixed (i.e., preserved) by conventional methodology. See, e.g., Lee G. Luna, HT (ASCP) Ed., 1960, Manual of Histological Staining Method of the Armed Forces Institute of Pathology 3^{rd} edition, The Blakston Division McGraw-Hill Book Company, New York; Ulreka V. Mikel, Ed., 1994, The Armed Forces Institute of Pathology Advanced Laboratory Methods in Histology and Pathology, Armed Forces Institute of Pathology, American Registry of Pathology, Washington, D.C. One of skill in the art will appreciate that the choice of a fixative is determined by the purpose for which the tissue is to be histologically stained or otherwise analyzed. One of skill in the art will also appreciate that the length of fixation depends upon the size of the tissue sample and the fixative used.

Generally, a tissue sample is first fixed and is then dehydrated through an ascending series of alcohols, infiltrated and embedded with paraffin or other sectioning media so that the tissue sample may be sectioned. Alternatively, one may section the tissue and fix the sections obtained. By way of example, the tissue sample may be embedded and processed in paraffin by conventional methodology according to conventional techniques described by the references provided above. Examples of paraffin that may be used include, but are not limited to, Paraplast, Broloid, and Tissuemay. Once the tissue sample is embedded, the sample may be sectioned by a microtome according to conventional techniques. Sections may have a thickness in a range of about three microns to about twelve microns, and preferably, a thickness in a range of about 5 microns to about 10 microns. In one aspect, a section may have an area of about 10 mm² to about 1 cm². Once cut, the sections may be attached to slides by several standard methods. Examples of slide adhesives include, but are not limited to, silane, gelatin and poly-L-lysine. Paraffin embedded sections may be attached to positively charged slides and/or slides coated with poly-L-lysine.

If paraffin has been used as the embedding material, the tissue sections are generally deparaffinized and rehydrated to water prior to detection of biomarkers. Tissue sections may be deparaffinized by several conventional standard methodologies. For example, xylenes and a gradually descending series of alcohols may be used according to conventional techniques described by the references provided above. Alternatively, commercially available deparaffinizing non-organic agents such as Hemo-De® (CMS, Houston, Tex.) may be used.

Mammalian tissue culture cells, or fresh or frozen tissues may be prepared by conventional cell lysis techniques (e.g., 0.14 M NaCl, 1.5 mM MgCl₂, 10 mM Tris-Cl (pH 8.6), 0.5% Nonidet P-40, and protease and/or phosphatase inhibitors as required). For fresh mammalian tissues, sample preparation may also include a tissue disaggregation step, such as crushing, mincing, grinding or sonication.

Many advantages are provided by measuring activated HER3 populations using releasable molecular tags, including (1) separation of released molecular tags from an assay mixture provides greatly reduced background and a significant gain in sensitivity; and (2) the use of molecular tags that are specially designed for ease of separation and detection provides a convenient multiplexing capability so that multiple receptor complex components may be readily measured simultaneously in the same assay. Assays employing such tags can have a variety of forms and are disclosed in the following references: U.S. Pat. Nos. 7,105,308 and 6,627,400; published U.S. Patent Application Nos. 2002/0013126, 2003/0170915, 2002/0146726, 2009/0191559, 2010/0143927, 2010/0233732, and 2010/0210034; and International Patent Publication No. WO 2004/011900, each of which are incorporated herein by reference in their entireties. For example, a wide variety of separation techniques may be employed that can distinguish molecules based on one or more physical, chemical or optical differences among molecules being separated including electrophoretic mobility, molecular weight, shape, solubility, pKa, hydrophobicity, charge, charge/mass ratio or polarity. In one aspect, molecular tags in a plurality or set differ in electrophoretic mobility and optical detection characteristics and are separated by electrophoresis. In another aspect, molecular tags in a plurality or set may differ in molecular weight, shape, solubility, pKa, hydrophobicity, charge, polarity and are separated by normal phase or reverse phase HPLC, ion exchange HPLC, capillary electrochromatography, mass spectroscopy or gas phase chromatography.

Sets of molecular tags are provided that can be separated into distinct bands or peaks by a separation technique after they are released from binding compounds. Identification and quantification of such peaks provides a measure or profile of the presence and/or amounts of proteins, protein complexes and post-translationally modified proteins. In some embodiments, molecular tags within a set may be chemically diverse. In other embodiments, sets of molecular tags may be chemically related. For example, molecular tags may all be peptides, may consist of different combinations of the same basic building blocks or monomers, or may be synthesized using the same basic scaffold with different substituent groups for imparting different separation characteristics. The number of molecular tags in a plurality may vary depending on several factors including the mode of separation employed, the labels used on the molecular tags for detection, the sensitivity of the binding moieties and the efficiency with which the cleavable linkages are cleaved.

Measurements made directly on tissue samples may be normalized by including measurements on cellular or tissue targets that are representative of the total cell number in the sample and/or the numbers of particular subtypes of cells in the sample. The additional measurement may be preferred, or even necessary, because of the cellular and tissue heterogeneity in patient samples, particularly tumor samples, which may comprise substantial fractions of normal cells.

As mentioned above, mixtures containing pluralities of different binding compounds may be provided, wherein each different binding compound has one or more molecular tags attached through cleavable linkages. The nature of the binding compound, cleavable linkage and molecular tag may vary widely. A binding compound may comprise an antibody binding composition, an antibody composition, an antibody, a peptide, a peptide or non-peptide ligand for a cell surface receptor, a protein, an oligonucleotide, an oligonucleotide analog, such as a peptide nucleic acid, a lectin or any other molecular entity that is capable of specifically binding to a target protein or molecule or stable complex formation with an analyte of interest, such as an activated HER3. In one aspect, a binding compound can be represented by the following formula:

B-(L-E)ₖ

wherein B is binding moiety; L is a cleavable linkage, and E is a molecular tag. In homogeneous assays, cleavable linkage, L, may be an oxidation-labile linkage, and more preferably, it is a linkage that may be cleaved by singlet oxygen. Alternatively, it may be a linkage that is sensitive to cleavage by reduction, for example by DTT. The moiety “-(L-E)ₖ” indicates that a single binding compound may have multiple molecular tags attached via cleavable linkages. In one aspect, k is an integer greater than or equal to one, but in other embodiments, k may be greater than several hundred, e.g., 100 to 500 or k is greater than several hundred to as many as several thousand, e.g., 500 to 5000. Usually each of the plurality of different types of binding compounds has a different molecular tag, E. Cleavable linkages, e.g., oxidation-labile linkages, and molecular tags, E, are attached to B by way of conventional chemistries.

In some embodiments, B is an antibody binding composition or antibody composition that specifically binds to a target, such as an antigenic determinant on HER3. Antibodies specific for HER3 epitopes are provided in the examples set forth herein. Antibody compositions are readily formed from a wide variety of commercially available antibodies, either monoclonal or polyclonal. In particular, antibodies specific for epidermal growth factor receptors are disclosed in U.S. Pat. Nos. 5,677,171; 5,772,997; 5,968,511; 5,480,968; 5,811,098, each of which are incorporated by reference in its entirety. U.S. Pat. No. 5,599,681, hereby incorporated by reference in its entirety, discloses antibodies specific for phosphorylation sites of proteins. Commercial vendors, such as e.g., Cell Signaling Technology (Beverly, Mass.), Biosource International (Camarillo, Calif.) and Upstate (Charlottesville, Va.), also provide monoclonal and polyclonal antibodies.

Cleavable linkage, L, can be virtually any chemical linking group that may be cleaved under conditions that do not degrade the structure or affect detection characteristics of the released molecular tag, E. In certain embodiments, a cleaving probe used in a homogeneous assay format, has a cleavable linkage, L, cleaved by a cleavage agent generated by the cleaving probe that acts over a short distance so that only cleavable linkages in the immediate proximity of the cleaving probe are cleaved. Typically, such an agent must be activated by making a physical or chemical change to the reaction mixture so that the agent produces a short lived active species that diffuses to a cleavable linkage to effect cleavage. In a homogeneous format, the cleavage agent is preferably attached to a binding moiety, such as an antibody, that targets prior to activation the cleavage agent to a particular site in the proximity of a binding compound with releasable molecular tags.

In a non-homogeneous format, because specifically bound binding compounds are separated from unbound binding compounds, a wider selection of cleavable linkages and cleavage agents are available for use. Cleavable linkages may not only include linkages that are labile to reaction with a locally acting reactive species, such as hydrogen peroxide, singlet oxygen or the like, but also linkages that are labile to agents that operate throughout a reaction mixture, such as base-labile linkages, photocleavable linkages, linkages cleavable by reduction, linkages cleaved by oxidation, acid-labile linkages and peptide linkages cleavable by specific proteases. References describing many such linkages include Greene and Wuts, 1991, Protective Groups in Organic Synthesis, Second Edition, John Wiley & Sons, New York; Hermanson, 1996, Bioconjugate Techniques, Academic Press, New York; and U.S. Pat. No. 5,565,324. Photocleavable linkages also include those disclosed in U.S. Pat. No. 5,986,076.

In some embodiments, commercially available cleavable reagent systems may be employed with the invention. In some embodiments, the cleaving probe comprises a compound that directly cleaves the cleavable linkage to release the tag. For example, a disulfide linkage may be introduced between an antibody binding composition and a molecular tag using a heterofunctional agent such as N-succinimidyl 3-(2-pyridyldithio)propionate (SPDP), succinimidyloxycarbonyl-\acute{α}-methyl-\acute{α}-(2-pyridyldithio) toluene (SMPT) or the like, available from vendors such as Pierce Chemical Company (Rockford, Ill.). Disulfide bonds introduced by such linkages can be broken by treatment with a reducing agent, such as dithiothreitol (DTT), dithioerythritol (DTE), 2-mercaptoethanol or sodium borohydride. Typical concentrations of reducing agents to effect cleavage of disulfide bonds are in the range of about 1 mM to 100 mM. An oxidatively labile linkage may be introduced between an antibody binding composition and a molecular tag using the homobifunctional NHS ester cross-linking reagent, disuccinimidyl tartarate (DST)(available from Pierce) that contains central cis-diols that are susceptible to cleavage with sodium periodate (e.g., 15 mM periodate at physiological pH for 4 hours). Linkages that contain esterified spacer components may be cleaved with strong nucleophilic agents, such as hydroxylamine, e.g., 0.1 N hydroxylamine, pH 8.5, for 3-6 hours at 37° C. Such spacers can be introduced by a homobifunctional cross-linking agent such as ethylene glycol bis(succinimidylsuccinate)(EGS) available from Pierce (Rockford, Ill.). A base labile linkage can be introduced with a sulfone group. Homobifunctional cross-linking agents that can be used to introduce sulfone groups in a cleavable linkage include bis[2-(succinimidyloxycarbonyloxy)ethyl]sulfone (BSOCOES), and 4,4-difluoro-3,3-dinitrophenyl-sulfone (DFDNPS). Exemplary basic conditions for cleavage include 0.1 M sodium phosphate, adjusted to pH 11.6 by addition of Tris base, containing 6 M urea, 0.1% SDS, and 2 mM DTT, with incubation at 37° C. for 2 hours.

When L is oxidation labile, L may be a thioether or its selenium analog; or an olefin, which contains carbon-carbon double bonds, wherein cleavage of a double bond to an oxo group, releases the molecular tag, E. Illustrative oxidation labile linkages are disclosed in U.S. Pat. Nos. 6,627,400 and 5,622,929 and in published U.S. Patent Application Nos. 2002/0013126 and 2003/0170915; each of which is hereby incorporated herein by reference in its entirety.

Molecular tag, E, in the present invention may comprise an electrophoric tag as described in the following references when separation of pluralities of molecular tags are carried out by gas chromatography or mass spectrometry: See, e.g., Zhang et al., 2002, Bioconjugate Chem. 13:1002-1012; Giese, 1983, Anal. Chem. 2:165-168; and U.S. Pat. Nos. 4,650,750; 5,360,819; 5,516,931; and 5,602,273, each of which is hereby incorporated by reference in its entirety.

Molecular tag, E, is preferably a water-soluble organic compound that is stable with respect to the active species, especially singlet oxygen, and that includes a detection or reporter group. Otherwise, E may vary widely in size and structure. In one aspect, E has a molecular weight in the range of about 50 to about 2500 daltons, more preferably, from about 50 to about 1500 daltons. E may comprise a detection group for generating an electrochemical, fluorescent or chromogenic signal. In embodiments employing detection by mass, E may not have a separate moiety for detection purposes. Preferably, the detection group generates a fluorescent signal.

Molecular tags within a plurality are selected so that each has a unique separation characteristic and/or a unique optical property with respect to the other members of the same plurality. In one aspect, the chromatographic or electrophoretic separation characteristic is retention time under a set of standard separation conditions conventional in the art, e.g., voltage, column pressure, column type, mobile phase or electrophoretic separation medium. In another aspect, the optical property is a fluorescence property, such as emission spectrum, fluorescence lifetime or fluorescence intensity at a given wavelength or band of wavelengths. Preferably, the fluorescence property is fluorescence intensity. For example, each molecular tag of a plurality may have the same fluorescent emission properties, but each will differ from one another by virtue of a unique retention time. On the other hand, one or two or more of the molecular tags of a plurality may have identical migration or retention times, but they will have unique fluorescent properties, e.g., spectrally resolvable emission spectra, so that all the members of the plurality are distinguishable by the combination of molecular separation and fluorescence measurement.

Preferably, released molecular tags are detected by electrophoretic separation and the fluorescence of a detection group. In such embodiments, molecular tags having substantially identical fluorescence properties have different electrophoretic mobilities so that distinct peaks in an electropherogram are formed under separation conditions. Preferably, pluralities of molecular tags of the invention are separated by conventional capillary electrophoresis apparatus, either in the presence or absence of a conventional sieving matrix. During or after electrophoretic separation, the molecular tags are detected or identified by recording fluorescence signals and migration times (or migration distances) of the separated compounds or by constructing a chart of relative fluorescent and order of migration of the molecular tags (e.g., as an electropherogram). Preferably, the presence, absence and/or amounts of molecular tags are measured by using one or more standards as disclosed by published U.S. Patent Application No. 2003/0170734A1, which is hereby incorporated by reference in its entirety.

Pluralities of molecular tags may also be designed for separation by chromatography based on one or more physical characteristics that include molecular weight, shape, solubility, pKa, hydrophobicity, charge, polarity or the like, e.g., as disclosed in published U.S. Pat. No. 7,255,999, which hereby is incorporated by reference in its entirety. A chromatographic separation technique is selected based on parameters such as column type, solid phase, mobile phase and the like, followed by selection of a plurality of molecular tags that may be separated to form distinct peaks or bands in a single operation. Several factors determine which HPLC technique is selected for use in the invention, including the number of molecular tags to be detected (i.e., the size of the plurality), the estimated quantities of each molecular tag that will be generated in the assays, the availability and ease of synthesizing molecular tags that are candidates for a set to be used in multiplexed assays, the detection modality employed and the availability, robustness, cost and ease of operation of HPLC instrumentation, columns and solvents. Generally, columns and techniques are favored that are suitable for analyzing limited amounts of sample and that provide the highest resolution separations. Guidance for making such selections can be found in the literature, such as, for example, Snyder et al., 1988, Practical HPLC Method Development, John Wiley & Sons, New York; Millner, 1999, High Resolution Chromatography: A Practical Approach, Oxford University Press, New York; Chi-San Wu, 1999, Column Handbook for Size Exclusion Chromatography, Academic Press, San Diego; and Oliver, 1989, HPLC of Macromolecules: A Practical Approach, Oxford University Press, Oxford, England.

In one aspect, molecular tag, E, is (M, D), where M is a mobility-modifying moiety and D is a detection moiety. The notation “(M, D)” is used to indicate that the ordering of the M and D moieties may be such that either moiety can be adjacent to the cleavable linkage, L. That is, “B-L-(M, D)” designates binding compound of either of two forms: “B-L-M-D” or “B-L-D-M.”

Detection moiety, D, may be a fluorescent label or dye, a chromogenic label or dye or an electrochemical label. Preferably, D is a fluorescent dye. Exemplary fluorescent dyes for use with the invention include water-soluble rhodamine dyes, fluoresceins, 4,7-dichlorofluoresceins, benzoxanthene dyes and energy transfer dyes, as disclosed in the following references: Handbook of Molecular Probes and Research Reagents, 8^{th} ed. (2002), Molecular Probes, Eugene, Oreg.; U.S. Pat. Nos. 6,191,278, 6,372,907, 6,096,723, 5,945,526, 4,997,928, and 4,318,846; and Lee et al., 1997, Nucleic Acids Research 25:2816-2822. Preferably, D is a fluorescein or a fluorescein derivative.

Once each of the binding compounds is separately derivatized by a different molecular tag, it is pooled with other binding compounds to form a plurality of binding compounds. Usually, each different kind of binding compound is present in a composition in the same proportion; however, proportions may be varied as a design choice so that one or a subset of particular binding compounds are present in greater or lower proportion depending on the desirability or requirements for a particular embodiment or assay. Factors that may affect such design choices include, but are not limited to, antibody affinity and avidity for a particular target, relative prevalence of a target, fluorescent characteristics of a detection moiety of a molecular tag and the like.

In some embodiments, a cleavage-inducing moiety, or cleaving agent, is a chemical group that produces an active species that is capable of cleaving a cleavable linkage, preferably by oxidation. Preferably, the active species is a chemical species that exhibits short-lived activity so that its cleavage-inducing effects are only in the proximity of the site of its generation. Either the active species is inherently short lived, so that it will not create significant background beyond the proximity of its creation, or a scavenger is employed that efficiently scavenges the active species, so that it is not available to react with cleavable linkages beyond a short distance from the site of its generation. Illustrative active species include singlet oxygen, hydrogen peroxide, NADH, and hydroxyl radicals, phenoxy radical, superoxide and the like. Illustrative quenchers for active species that cause oxidation include polyenes, carotenoids, vitamin E, vitamin C, amino acid-pyrrole N-conjugates of tyrosine, histidine and glutathione. See, e.g., Beutner et al., 2000, Meth. Enzymol. 319:226-241.

One consideration in designing assays employing a cleavage-inducing moiety and a cleavable linkage is that they not be so far removed from one another when bound to a receptor complex that the active species generated by the cleavage-inducing moiety cannot efficiently cleave the cleavable linkage. In one aspect, cleavable linkages preferably are within about 1000 nm and preferably within about 20-200 nm, of a bound cleavage-inducing moiety. More preferably, for photosensitizer cleavage-inducing moieties generating singlet oxygen, cleavable linkages are within about 20-100 nm of a photosensitizer in a receptor complex. The range within which a cleavage-inducing moiety can effectively cleave a cleavable linkage (that is, cleave enough molecular tag to generate a detectable signal) is referred to herein as its “effective proximity.” One of ordinary skill in the art will recognize that the effective proximity of a particular sensitizer may depend on the details of a particular assay design and may be determined or modified by routine experimentation.

A sensitizer is a compound that can be induced to generate a reactive intermediate, or species, usually singlet oxygen. Preferably, a sensitizer used in accordance with the invention is a photosensitizer. Other sensitizers included within the scope of the invention are compounds that on excitation by heat, light, ionizing radiation or chemical activation will release a molecule of singlet oxygen. The best known members of this class of compounds include the endoperoxides such as 1,4-biscarboxyethyl-1,4-naphthalene endoperoxide, 9,10-diphenylanthracene-9,10-endoperoxide and 5,6,11,12-tetraphenyl naphthalene 5,12-endoperoxide. Heating or direct absorption of light by these compounds releases singlet oxygen. Further sensitizers are disclosed by Di Mascio et al., 1994, FEBS Lett. 355:287; and Kanofsky, 1983, J. Biol. Chem. 258:5991-5993; Pierlot et al., 2000, Meth. Enzymol. 319:3-20.

Photosensitizers may be attached directly or indirectly, via covalent or non-covalent linkages, to the binding agent of a class-specific reagent. Guidance for constructing such compositions, particularly for antibodies as binding agents are available in the literature, e.g., in the fields of photodynamic therapy, immunodiagnostics, and the like. Exemplary guidance may be found in Ullman et al., 1994, Proc. Natl. Acad. Sci. USA 91, 5426-5430; Strong et al., 1994, Ann. New York Acad. Sci. 745: 297-320; Yarmush et al., 1993, Crit. Rev. Therapeutic Drug Carrier Syst. 10: 197-252; and U.S. Pat. Nos. 5,340,716, 5,516,636, 5,709,994, and 6,251,581.

A large variety of light sources are available to photo-activate photosensitizers to generate singlet oxygen. Both polychromatic and monochromatic sources may be used as long as the source is sufficiently intense to produce enough singlet oxygen in a practical time duration. The length of the irradiation depends on the nature of the photosensitizer, the nature of the cleavable linkage, the power of the source of irradiation and its distance from the sample. In general, the period for irradiation may be more than about a microsecond to as long as about 10 minutes, usually in the range of about one millisecond to about 60 seconds. The intensity and length of irradiation should be sufficient to excite at least about 0.1% of the photosensitizer molecules, usually at least about 30% of the photosensitizer molecules and preferably, substantially all of the photosensitizer molecules. Exemplary light sources include lasers such as, e.g., helium-neon lasers, argon lasers, YAG lasers, He/Cd lasers and ruby lasers; photodiodes; mercury, sodium and xenon vapor lamps; incandescent lamps such as, e.g., tungsten and tungsten/halogen and flashlamps. An exemplary photoactivation device suitable for use in the methods of the invention is disclosed International Patent Publication No. WO 03/051669. In such embodiments, the photoactivation device is an array of light emitting diodes (LEDs) mounted in housing that permits the simultaneous illumination of all the wells in a 96-well plate.

Examples of photosensitizers that may be utilized in the present invention are those that have the above properties and those disclosed by U.S. Pat. Nos. 5,536,834, 5,763,602, 5,565,552, 5,709,994, 5,340,716, 5,516,636, 6,251,581, and 6,001,673; published European Patent Application No. 0484027; Martin et al., 1990, Methods Enzymol. 186:635-645; and Yarmush et al., 1993, Crit. Rev. Therapeutic Drug Carrier Syst. 10:197-252. As with sensitizers, in certain embodiments, a photosensitizer may be associated with a solid phase support by being covalently or non-covalently attached to the surface of the support or incorporated into the body of the support. In general, the photosensitizer is associated with the support in an amount necessary to achieve the necessary amount of singlet oxygen. Generally, the amount of photosensitizer is determined empirically according to routine methods.

In one embodiment, a photosensitizer is incorporated into a latex particle to form photosensitizer beads, e.g., as disclosed by U.S. Pat. Nos. 5,709,994 and 6,346,384; and International Patent Publication No. WO 01/84157. Alternatively, photosensitizer beads may be prepared by covalently attaching a photosensitizer, such as rose bengal, to 0.5 micron latex beads by means of chloromethyl groups on the latex to provide an ester linking group, as described in J. Amer. Chem. Soc., 97:3741 (1975). Methods employing latex particle, photosensitizer beads may be carried out, for example, in a conventional 96-well or 384-well microtiter plate, or the like, having a filter membrane that forms one wall, e.g., the bottom, of the wells that allows reagents to be removed by the application of a vacuum. This allows the convenient exchange of buffers, if the buffer required for specific binding of binding compounds is different than the buffer required for either singlet oxygen generation or separation. For example, in the case of antibody binding compounds or antibody compositions, a high salt buffer is required. If electrophoretic separation of the released tags is employed, then better performance is achieved by exchanging the buffer for one that has a lower salt concentration suitable for electrophoresis.

As an example, a cleaving probe may comprise a primary haptenated antibody and a secondary anti-hapten binding protein derivatized with multiple photosensitizer molecules. A preferred primary haptenated antibody is a biotinylated antibody and preferred secondary anti-hapten binding proteins may be either an anti-biotin antibody or streptavidin. Other combinations of such primary and secondary reagents are well known in the art. Exemplary combinations of such reagents are taught by Haugland, 2002, Handbook of Fluorescent Probes and Research Reagents, Ninth Edition, Molecular Probes, Eugene, Oreg. An exemplary combination of such reagents is described below. There binding compounds having releasable tags (“mT₁” and “mT₂”), and primary antibody derivatized with biotin are specifically bound to different epitopes of receptor dimer in membrane. Biotin-specific binding protein, e.g., streptavidin, is attached to biotin bringing multiple photosensitizers into effective proximity of binding compounds. Biotin-specific binding protein may also be an anti-biotin antibody and photosensitizers may be attached via free amine group on the protein by conventional coupling chemistries, e.g., Hermanson (supra). An exemplary photosensitizer for such use is an NHS ester of methylene blue prepared as disclosed in published European Patent Application 0510688.

The following general discussion of methods and specific conditions and materials are by way of illustration and not limitation. One of skill in the art will understand how the methods described herein can be adapted to other applications, particularly with using different samples, cell types, and target complexes.

In conducting the methods of the invention, a combination of the assay components is made, including the sample being tested, the binding compounds and optionally the cleaving probe. Generally, assay components may be combined in any order. In certain applications, however, the order of addition may be relevant. For example, one may wish to quantitatively monitor competitive binding or monitor the stability of an assembled complex. In such applications, reactions may be assembled in stages.

The amounts of each reagent can generally be determined empirically. The amount of sample used in an assay will be determined by the predicted number of target complexes present and the means of separation and detection used to monitor the signal of the assay. In general, the amounts of the binding compounds and the cleaving probe can be provided in molar excess relative to the expected amount of the target molecules in the sample, generally at a molar excess of at least about 1.5, more desirably about 10-fold excess, or more. In specific applications, the concentration used may be higher or lower, depending on the affinity of the binding agents and the expected number of target molecules present on a single cell. Where one is determining the effect of a chemical compound on formation of oligomeric cell surface complexes, the compound may be added to the cells prior to, simultaneously with, or after addition of the probes, depending on the effect being monitored.

The assay mixture can be combined and incubated under conditions that provide for binding of the probes to the cell surface molecules, usually in an aqueous medium, generally at a physiological pH (comparable to the pH at which the cells are cultures), maintained by a buffer at a concentration in the range of about 10 to 200 mM. Conventional buffers may be used, as well as other conventional additives as necessary, such as salts, growth medium, stabilizers, etc. Physiological and constant temperatures are normally employed. Incubation temperatures normally range from about 4° to 70° C., usually from about 15° to 45° C., more usually about 25° to 37° C.

After assembly of the assay mixture and incubation to allow the probes to bind to cell surface molecules, the mixture can be treated to activate the cleaving agent to cleave the tags from the binding compounds that are within the effective proximity of the cleaving agent, releasing the corresponding tag from the cell surface into solution. The nature of this treatment will depend on the mechanism of action of the cleaving agent. For example, where a photosensitizer is employed as the cleaving agent, activation of cleavage can comprise irradiation of the mixture at the wavelength of light appropriate to the particular sensitizer used.

Following cleavage, the sample can then be analyzed to determine the identity of tags that have been released. Where an assay employing a plurality of binding compounds is employed, separation of the released tags will generally precede their detection. The methods for both separation and detection are determined in the process of designing the tags for the assay. A preferred mode of separation employs electrophoresis, in which the various tags are separated based on known differences in their electrophoretic mobilities.

As mentioned above, in some embodiments, if the assay reaction conditions may interfere with the separation technique employed, it may be necessary to remove, or exchange, the assay reaction buffer prior to cleavage and separation of the molecular tags. For example, assay conditions may include salt concentrations (e.g., required for specific binding) that degrade separation performance when molecular tags are separated on the basis of electrophoretic mobility. Thus, such high salt buffers may be removed, e.g., prior to cleavage of molecular tags, and replaced with another buffer suitable for electrophoretic separation through filtration, aspiration, dilution or other means.

In certain embodiments, the subject may be administered a combination therapy that includes a HER3-acting agent. The combination therapy can include the HER3-acting agent in combination with one or more of any chemotherapeutic agent known to one of skill in the art without limitation. Preferably, the chemotherapeutic agent has a different mechanism of action from the HER3-acting agent. For example, the chemotherapeutic agent can be an anti-metabolite (e.g., 5-flourouricil (5-FU), methotrexate (MTX), fludarabine, etc.), an antimicrotubule agent (e.g., vincristine; vinblastine; taxanes such as paclitaxel and docetaxel; etc.), an alkylating agent (e.g., cyclophosphamide, melphalan, bischloroethylnitrosurea, etc.), platinum agents (e.g., cisplatin, carboplatin, oxaliplatin, JM-216, CI-973, etc.), anthracyclines (e.g., doxorubicin, daunorubicin, etc.), antibiotic agents (e.g., mitomycin-C, actinomycin D, etc.), topoisomerase inhibitors (e.g., etoposide, camptothecins, etc.) or other any other chemotherapeutic agents known to one skilled in the art.

Particular examples of chemotherapeutic agents that can be used in the various embodiments of the invention, including pharmaceutical compositions, dosage forms, and kits of the invention, include, without limitation, cytarabine, melphalan, topotecan, fludarabine, etoposide, idarubicin, daunorubicin, mitoxantrone, cisplatin paclitaxel, and cyclophosphamide.

Other chemotherapeutic agents that may be used include abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate.

In another aspect, the invention is drawn to a method for determining whether a subject with a HER3 activated cancer is unlikely to respond to treatment with at least one chemotherapeutic agent in addition to a HER3-acting agent and/or the patient is likely to have a short time course. In certain embodiments, the method comprises measuring in a biological sample from the subject's cancer an amount of HER3 and/or activated HER3, wherein if the level of HER3 and/or activated HER3 is high or very high, then the patient is unlikely to respond to at least one chemotherapeutic agent in addition to a HER3 acting agent.

In another aspect, the invention is drawn to a method for determining whether a subject with a HER3 positive cancer is likely to respond to treatment with at least one chemotherapeutic agent in addition to a HER3-acting agent. In certain embodiments, the method comprises measuring in a biological sample from the subject's cancer an amount of activated HER3, wherein if the level of activated HER3 is high, then the patient is likely to respond to at least one chemotherapeutic agent in addition to the HER3-acting agent. In certain embodiments, the biological sample comprises FFPE tissue samples. In certain embodiments, the cancer is metastatic or recurrent. In some embodiments, any cancer that may be sensitive to a HER3-acting agent may be monitored. The HER3-acting agent may be any HER3-acting agent. In certain embodiments, the HER3-acting agent is one of the agents described herein. Alternatively, other additional chemotherapeutic agents as known in the art may be evaluated. In certain embodiments, likeliness to respond or time course is measured with respect to overall survival rate, time to progression and/or using the RECIST criteria.

### Systems Utilizing Activated HER3 Measurements

In another aspect, the invention comprises systems comprising a first computing device, the first computing device in communication with a database; a first application executing on the first computing device, the first application configured to receive a plurality of laboratory test results for a plurality of subjects and store the plurality of laboratory test results in the database, wherein the plurality of laboratory test results comprise an amount of total HER3 and at least one of an amount of HER2-HER3 heterodimer, phosphorylated HER3, or HER3/PI3K complex measured in a tumor sample from a subject; a second computing device, the second computing device in communication with the database; and a second application executing on the second computing device, the second application configured to: query the database for laboratory test results for a subject from the plurality of subject; receive the laboratory test results for the subject from the database; determine a test result based at least in part on the received laboratory test results for the subject, the test results comprising the amount of total HER3 and the ratio of at least one of HER2-HER3 heterodimer, phosphorylated HER3, or HER3/PI3K complex to total HER3 protein in a tumor sample obtained from the subject; generate a test result report for the subject, the test result report comprising the amount of activated HER3 in the tumor sample and based at least in part on the test result for the subject; and transmit the test result report for the patient to a third computing device.

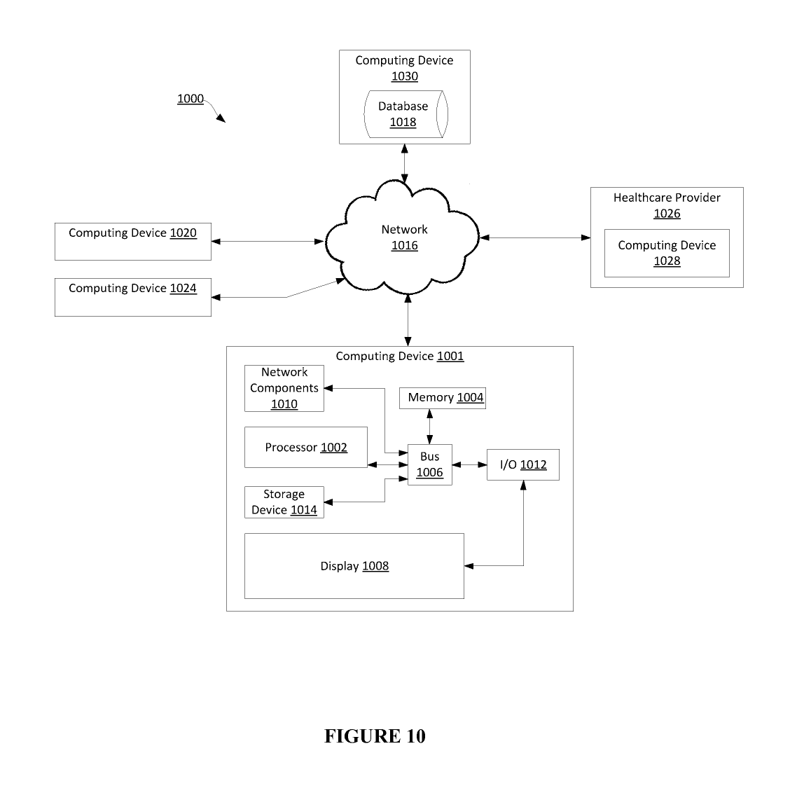

In some embodiments, the invention comprises a system, for example, the system 1000 shown in FIG. 10. The system 1000 includes various components. As used herein, the term “component” is broadly defined and includes any suitable apparatus or collections of apparatuses suitable for carrying out the recited method. The components need not be integrally connected or situated with respect to each other in any particular way. Embodiments include any suitable arrangements of the components with respect to each other. For example, the components need not be in the same room. But in some embodiments, the components are connected to each other in an integral unit. In some embodiments, the same components may perform multiple functions.

The system 1000 may comprise one or more computing devices 1001. Typical examples of computing devices 1001 include a general-purpose computer, a printer, a programmed microprocessor, a microcontroller, a peripheral integrated circuit element, and other devices or arrangements of devices that are capable of implementing the steps that constitute the method of the present technique.

The computing device 1001 comprises a memory 1004. The memory 1004 may include random access memory (RAM) and read only memory (ROM), as well as removable media devices, memory cards, flash cards, etc. The computing device 1001 may further comprise a storage device 1014. The storage device 1014 can be a hard disk drive or a removable storage drive such as a floppy disk drive, optical disk drive, etc. The storage device 1014 can also be other similar means for loading computer programs or other instructions into the computing device 1001.

A computing device 1001 also comprises a processor 1002. The processor 1002 executes a set of instructions that are stored in one or more storage elements (e.g., memory 1004 or storage device 1014), in order to process input data. The storage elements may also hold data or other information as desired. The storage elements may be in the form of an information source or a physical memory 1004 element present in the processing machine.

A computing device 1001 typically will include an operating system that provides executable program instructions for the general administration and operation of that computing device 1001, and typically will include a computer-readable storage medium (e.g., a hard disk, random access memory, read only memory, etc.) storing instructions that, when executed by a processor of the server, allow the computing device 1001 to perform its intended functions. Suitable implementations for the operating system and general functionality of the computing device 1001 are known or commercially available, and are readily implemented by persons having ordinary skill in the art, particularly in light of the disclosure herein.

As discussed above, some embodiments comprise a processor 1002 which is configured to execute computer-executable program instructions and/or to access information stored in memory 1004. The instructions may comprise processor-specific instructions generated by a compiler and/or an interpreter from code written in any suitable computer-programming language including, for example, C, C++, C#, Visual Basic, Java, Python, Perl, JavaScript, and ActionScript (Adobe Systems, Mountain View, Calif.). The set of instructions for execution by the computing device 1001 may include various commands that instruct the processor 1002 to perform specific tasks such as the steps that constitute the method of the present technique. The set of instructions may be in the form of a software program. Further, the software may be in the form of a collection of separate programs, a program module with a larger program or a portion of a program module, as in the present technique. The software may also include modular programming in the form of object-oriented programming. The processing of input data by the processing machine may be in response to user commands, results of previous processing, or a request made by another processing machine.

In some embodiments, the computing device 1001 may comprise a single processor 1002. In other embodiments, the computing device 1001 comprises two or more processors. Such processors 1002 may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices. The processor 1002 is connected to a communication bus 1006. The communication bus 1006 may be connected to one or more other components, for example, the processor 1002, an input device (e.g., a mouse, keyboard, controller, touch screen, or keypad), and an output device (e.g., a display 1008, printer, or speaker).

The computing device 1001 can also include network components 1010. The network components 1010 allow the computing device 1001 to connect to one or more networks 1016 and/or other databases (e.g., database 1018) through an I/O interface. Although depicted in FIG. 10 as a single network 1016, the network 1016 can include any number of networks. The network components 1010 allow the transfer to, as well as reception of data from, a network 1016 and/or databases. The network components 1010 may include a modem, an Ethernet card, or any similar device which enables the computing device 1001 to connect to databases and/or networks 1016 such as LAN, MAN, WAN and the Internet. The network components 1010 may comprise a network interface. In some embodiments, the network interface is configured for communicating via wired or wireless communication links. For example, the network interface 1010 may allow for communication over networks via Ethernet, IEEE 802.11 (Wi-Fi), 802.16 (Wi-Max), Bluetooth, infrared, etc. As another example, the network interface may allow for communication over networks such as CDMA, GSM, UMTS, or other cellular communication networks. In some embodiments, the network interface 1010 may allow for point-to-point connections with another device, such as via the Universal Serial Bus (USB), 1394 FireWire, serial or parallel connections, or similar interfaces. Some embodiments of suitable computing devices 1001 may comprise two or more network interfaces 1010 for communication over one or more networks. In some embodiments, the computing device may include a database 1018 in addition to or in place of a network interface 1010.

The system 1000 can include a variety of data stores and other memory and storage media as discussed above. These can reside in a variety of locations, such as on a storage medium local to (and/or resident in) one or more of the computing devices 1001 or remote from any or all of the computing devices 1001 across the network 1016. In a particular set of embodiments, the information may reside in a storage-area network (SAN) familiar to those skilled in the art. Similarly, any necessary files for performing the functions attributed to the computers, servers, or other network devices may be stored locally and/or remotely, as appropriate.

Computing device 1001 can also include a computer-readable storage media reader. The computer-readable storage media reader can be connected with, or configured to receive, a computer-readable storage medium, representing remote, local, fixed, and/or removable storage devices as well as storage media for temporarily and/or more permanently containing, storing, transmitting, and retrieving computer-readable information. The system 1000 and various devices also typically will include a number of software applications, modules, services, or other elements located within at least one working memory device, including an operating system and application programs, such as a client application or Web browser. It should be appreciated that alternate embodiments may have numerous variations from that described above. For example, customized hardware might also be used and/or particular elements might be implemented in hardware, software (including portable software, such as applets), or both. Further, connection to other computing devices 1020, 1040, such as network input/output devices, may be employed.

Storage media and computer readable media for containing code, or portions of code, can include any appropriate media known or used in the art, including storage media and communication media, such as but not limited to volatile and non-volatile, removable and non-removable media implemented in any method or technology for storage and/or transmission of information such as computer readable instructions, data structures, program modules, or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices, or any other medium which can be used to store the desired information and which can be accessed by the a system 1000 device. Based on the disclosure and teachings provided herein, a person of ordinary skill in the art will appreciate other ways and/or methods to implement the various embodiments.

A computer-readable medium may comprise, but is not limited to, an electronic, optical, magnetic, or other storage device capable of providing a processor with computer-readable instructions. Other examples include, but are not limited to, a floppy disk, CD-ROM, DVD, magnetic disk, memory chip, ROM, RAM, SRAM, DRAM, content-addressable memory (CAM), DDR, flash memory such as NAND flash or NOR flash, an ASIC, a configured processor, optical storage, magnetic tape or other magnetic storage, or any other medium from which a computer processor can read instructions. In one embodiment, the computing device 1001 may comprise a single type of computer-readable medium such as random access memory (RAM). In other embodiments, the computing device 1001 may comprise two or more types of computer-readable medium such as random access memory (RAM), a disk drive, and cache. The computing device 1001 may be in communication with one or more external computer-readable mediums such as an external hard disk drive or an external DVD drive.

The computing device 1001 may further include I/O components 1012, which may be used to facilitate wired or wireless connection to input and output devices. Some embodiments of suitable computing devices 1001 may comprise or be in communication with a number of external or internal devices such as a mouse, a CD-ROM, DVD, a keyboard, a display, audio speakers, one or more microphones, or any other input or output devices. For example, the computing device may be in communication with various user interface devices and a display 1008. The display 1008 may use any suitable technology including, but not limited to, LCD, LED, CRT, and the like.

The system 1000 may include any number of computing devices 1001. For example, in one embodiment, the system 1000 includes one computing device 1001. In the example shown in FIG. 10, the system 1000 includes a plurality of computing devices 1001, 1020, 1024, 1028, 1030. The computing devices may be of the same or different types. For example, in some embodiments, computing device 1001 may comprise a desktop computer, while computing device 1024 may comprise a printer. Further, in some embodiments, the computing devices may be at the same or different locations. For example, in the embodiment shown in FIG. 10, one computing device 1001 may be located onsite at a testing center, while another computing device 1028 may be located offsite at a healthcare provider's office.

The system 1000 further includes a database 1018. The one or more computing devices 1001 are in communication with the database 1018. In some embodiments, the database may comprise, for example, a MySQL database. The database 1018 may contain data that may be retrievable by one or more computing devices (e.g., computing device 1001, 1020 or 1028). In some embodiments, the database 1018 may itself be a part of a computing device 1030. The database 1018 may comprise data related to subject information, subject sample information, reference population, cancer treatment options, healthcare provider information, laboratory test results, and laboratory test reports. For example, data related to subjects may include subject names, addresses, telephone numbers, subject identification numbers, providers to which the subjects are associated, medical history (including, e.g., disease status such as cancer status, HER2 status, prior test results), medications and/or treatments, relatives, health care provider plans, account balances, access information, or other information related to one or more subjects. Data related to subject samples may include subject names, addresses, telephone numbers, subject identification numbers, date of collection, sample type (e.g., nature of collection, preparation method), disease status (e.g., type of cancer, including HER2 status or other biomarker status; other conditions), central testing laboratory results, date of processing/analysis of samples, laboratory test results, providers to which the subjects are associated, medical history (including, e.g., disease status such as cancer status, HER2 status, prior test results), medications and/or treatments, relatives, health care provider plans, account balances, access information, or other information related to one or more subjects. Data related to the reference population can include reference population subject information, reference population subject sample information, and information relating to clinical study participation, the median amount of total HER3 and at least one of the median amount of HER2-HER3 heterodimer, phosphorylated HER3, or HER3/PI3K complex measured in tumor samples from reference population subjects, and the median ratio of at least one of HER2-HER3 heterodimer, phosphorylated HER3, or HER3/PI3K complex to total HER3 protein in tumor samples obtained from reference population subjects. Data for each subject individually and for the reference population as a whole may be included. In some embodiments, the reference population comprises subjects with the same type of cancer as the subject. Data related to healthcare providers can include names, addresses, phone numbers, personnel, usernames, passwords, other security information, access levels, and other information associated with one or more providers. Data related to laboratory test results can include the amount of total HER3 and at least one of an amount of HER2-HER3 heterodimer, phosphorylated HER3, or HER3/PI3K complex measured in tumor samples from the plurality of subjects, and may also include the ratio of at least one of HER2-HER3 heterodimer, phosphorylated HER3, or HER3/PI3K complex to total HER3 protein in a tumor sample obtained from the subject. Data related to test result reports can include the amount of activated HER3 in the tumor sample and based at least in part on the test result for the subject, including the amount of total HER3 and at least one of an amount of HER2-HER3 heterodimer, phosphorylated HER3, or HER3/PI3K complex measured in tumor samples from the plurality of subjects, the ratio of at least one of HER2-HER3 heterodimer, phosphorylated HER3, or HER3/PI3K complex to total HER3 protein in tumor samples obtained from the plurality of subjects, and reference population information.

In some embodiments, the system 1000 may execute one or more applications. The one or more applications may be executed on any number of computing devices (e.g., computing devices 1001, 1020, 1024, 1028, or 1030). In some embodiments, the system 1000 may execute an application configured to receive a plurality of laboratory test results. In some embodiments, the plurality of laboratory test results may be for a plurality of subjects. In other embodiments, the plurality of laboratory test results may be for a single subject. The system 1000 may store the plurality of laboratory test results in the database 1018.

The system 1000 may also execute an application configured to query the database 1018 for laboratory test results associated with one or more subjects. The system 1000 may determine a test result based at least in part on the received laboratory test results for the one or more subjects. In some embodiments, the test result may comprise the amount of total HER3 and the amount of at least one of HER2-HER3 heterodimer, phosphorylated HER3, or HER3/PI3K complex measured in a tumor sample obtained from the subject. In some embodiments, the test result may comprise the amount of total HER3 and the ratio of at least one of HER2-HER3 heterodimer, phosphorylated HER3, or HER3/PI3K complex to total HER3 protein measured in a tumor sample obtained from the subject.

In some embodiments, based on the test results, the system 1000 may generate a test result report for the one or more subjects. The test result report may comprise, for example, the amount of activated HER3 in a tumor sample. In some embodiments, the test result report may comprise the amount of activated HER3 in the tumor sample and be based at least in part on the test result for the one or more subjects. For example, in some embodiments, the test result report may include the amount of total HER3 and at least one of HER2-HER3 heterodimer, phosphorylated HER3, or HER3/PI3K complex measured in a tumor sample obtained from the one or more subjects, the ratio of at least one of HER2-HER3 heterodimer, phosphorylated HER3, or HER3/PI3K complex to total HER3 protein measured in a tumor sample obtained from the one or more subjects, and/or the the median amount of HER3 and the median ratio of at least one of HER2-HER3 heterodimer, phosphorylated HER3, or HER3/PI3K complex to total HER3 protein in the reference population of subjects having the same type of cancer as the subject. In some embodiments, the test result report is generated by comparing the amount of total HER3 and the ratio of at least one of HER2-HER3 heterodimer, phosphorylated HER3, or HER3/PI3K complex in the tumor sample obtained from the subject to the median amount of HER3 and the median ratio of at least one of HER2-HER3 heterodimer, phosphorylated HER3, or HER3/PI3K complex to total HER3 protein in a reference population of subjects. In some embodiments, the reference population comprises subjects with the same type of cancer as the subject.

In some embodiments, the test result report comprises information that the tumor sample from the subject has a high amount of activated HER3 if (i) the amount of total HER3 in the sample is above the median amount of total HER3 of a reference population and (ii) the ratio of at least one of HER2-HER3 heterodimer to HER3 total, phosphorylated HER3 to total HER3 in the sample, or HER3/PI3K complex is above the median ratio of at least one of HER2-HER3 heterodimer to HER3 total, phosphorylated HER3 to total HER3, or or HER3/PI3K complex to total HER3 in the reference population. In some embodiments, the test result report comprises information that the subject is more likely to respond to the HER3-targeted therapy if the tumor sample from the subject is characterized as having a high level of activated HER3, wherein a high level of activated HER3 comprises (i) the amount of total HER3 in the tumor sample from the subject being above the median amount of total HER3 of a reference population of subjects having the same type of cancer as the subject and (ii) the ratio of at least one of the amount of HER2-HER3 homodimer to the amount of HER3 total, the amount of phosphorylated HER1 to the amount of total HER3, or the amount of HER3-PI3K complex to the amount of HER3 total in the tumor sample being above the median ratio of at least one of the amount of HER2-HER3 homodimer to the amount of HER3 total, the amount of phosphorylated HER3 to the amount of total HER3, or the amount of HER3-PI3K complex to the amount of HER3 total, in the reference population of subjects having the same type of cancer as the subject. In some embodiments, the test result report may include information about HER3-targeted drugs.

In some embodiments, test result report may further comprise the amount of total HER2 protein in measured in the sample from the subject. The test result report may then comprises information of whether the subject has a HER2 positive cancer or a HER2 negative cancer. In some embodiments, the HER2 negative cancers and HER2 positive cancers have been characterized by immunohistochemical or in situ hybridization analysis (e.g., at a centralized testing laboratory). In some embodiments, the test result report may further comprise information that a co-therapy comprising a HER2-targeted therapy and a HER3-targeted therapy would be an appropriate therapy if the subject has a HER2 positive cancer. In some embodiments, the test result report may further comprise information indicating that the subject is more likely to respond to a co-therapy of a HER2-targeted agent and a HER3-targeted agent if the subject has a HER2 positive cancer. In certain embodiments, the test result report may further comprise information of whether the amount of total HER2 protein in the subject sample is below a first cutoff comprising a level of total HER2 protein corresponding to the bottom 5^{th} percentile of total HER2 protein expression in a reference population of HER2 positive cancers (i.e., HERmark® HER2 negative), if the amount of total HER2 protein is above a second cutoff comprising a level of total HER2 protein corresponding to a top 95^{th} percentile of total HER2 protein expression in a reference population of HER2 negative cancers (i.e., HERmark® HER2 positive), or whether the amount of total HER2 protein is above the first cutoff but below the second cutoff (i.e., HERmark® HER2 positive). In certain embodiments, the test result report may further comprise information that a co-therapy comprising a HER2-targeted therapy and a HER3-targeted therapy would be an appropriate treatment for the subject if the amount of total HER2 protein in the tumor sample is above the first cutoff. In certain embodiments, the test result report may further comprise information indicating that the subject is more likely to respond to a co-therapy of a HER2-targeted agent and a HER3-targeted agent if the amount of total HER2 protein in the biological sample is above the first cutoff. In some embodiments, the test result report may include information about HER3-targeted drugs and/or HER2-targeted drugs.

In some embodiments, the system 1000 may transmit the test result report. In some embodiments, the system 1000 may transmit the test report to a computing device, for example, computing device 1020. In some embodiments, the system 1000 may transmit the test result report to one or more recipients. In some embodiments, the recipient may be the subject or a healthcare provider. In some embodiments, the system may transmit the test result report via e-mail (e.g., to an e-mail account associated with the subject's healthcare provider), SMS, or text message.

In some embodiments, the system 1000 may store the test result report in the database 1018. Further, the system 1000 may provide an electronic notification to a computing device 1028. The computing device 1028 may be associated with a healthcare provider 1026, which may be associated with the subject. In some embodiments, the electronic notification may comprise an e-mail, a text message, or a push notification. The electronic notification may indicate that a test report is available, for example, for download from the database 1018.

### Methods Utilizing Activated HER3 Measurements

A further aspect of the invention comprises methods comprising receiving a plurality of laboratory test results in a database, wherein the plurality of laboratory test results comprise an amount of total HER3 and at least one of an amount of HER2-HER3 heterodimer, phosphorylated HER3, or HER3/PI3K complex measured in a tumor sample from a subject; storing the plurality of laboratory test results in the database; querying the database for laboratory test results for a subject from the plurality of subjects; receiving the laboratory test results for the subject from the database; determining a test result based in part on the received laboratory test results for the subject, the test result comprising the amount of total HER3 and the ratio of at least one of HER2-HER3 heterodimer, phosphorylated HER3, or HER3/PI3K complex to total HER3 protein in a tumor sample obtained from the subject; generating a test result report for the subject, the test result report comprising the amount of activated HER3 in the tumor sample and based at least in part on the test result for the subject; and transmitting the test result report for the subject to a computing device.

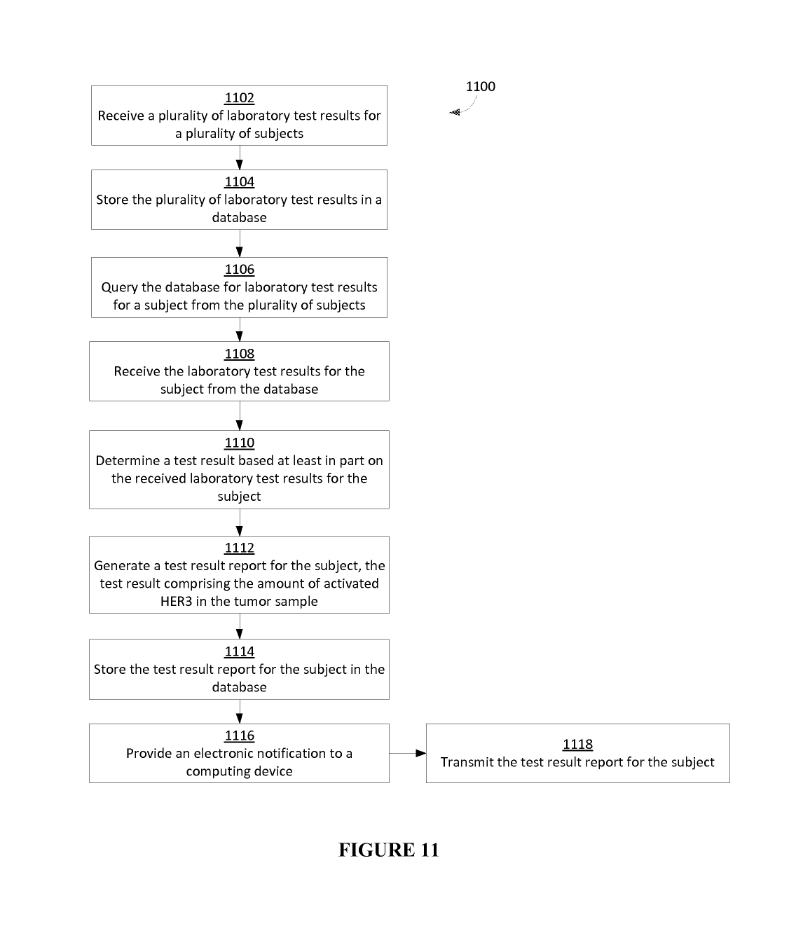

FIG. 11 is a flow chart of steps for performing a method for facilitating diagnosis, prognosis and treatment of cancer by detecting HER3 activation according to one embodiment. In some embodiments, the steps in FIG. 11 may be implemented in program code that is executed by a processor, for example, the processor in a general purpose computer, a mobile device, or a server. In some embodiments, these steps may be implemented by a group of processors. In some embodiments one or more steps shown in FIG. 11 may be omitted or performed in a different order. Similarly, in some embodiments, additional steps not shown in FIG. 11 may also be performed. The steps below are described with reference to components described above with regard to system 1000 shown in FIG. 10.

The method 1100 begins at step 1102 when processor 1002 receives a plurality of laboratory test results for a plurality of subjects. In some embodiments, the plurality of laboratory test results may comprise an amount of total HER3 and at least one of an amount of HER2-HER3 heterodimer, phosphorylated HER3, or HER3/PI3K complex measured in tumor samples from the plurality of subjects.

The method 1100 continues at step 1104 when processor 1002 stores the plurality of laboratory test results in a database 1018. In some embodiments, the processor 1002 may be in communication with the database 1018 via a LAN, WAN, or the Internet. In other embodiments, the database 1018 may be internal to the computing device 1001 housing the processor 1002, and the processor 1002 may be in communication with the database 1018 via a bus 1006 or other hardware configuration. The processor 1002 may transmit data associated with the plurality of laboratory test results to the database 1018.

The method 1100 continues at step 1106 when processor 1002 queries the database 1018 for laboratory test results for a subject from the plurality of subjects. The processor 1002 may query the database by transmitting one or more commands to the database 1018 (and/or a computing device 1030 housing the database 1018). The computing device 1030 housing the database 1018 may perform one or more steps to retrieve the laboratory test result data from the database 1018. Further, the computing device 1030 housing the database 1018 may transmit the queried laboratory test result data to the processor 1002.

The method 1100 continues at step 1108 when processor 1002 receives the laboratory test results for the subject from the database 1018. In some embodiments, the processor 1002 may be receive the laboratory test result data via a LAN, WAN, or Internet connection. In some embodiments, the processor 1002 may store the laboratory test result data in memory 1004.

The method 1100 continues at step 1110 when processor 1002 determines a test result based at least in part on the received laboratory test results for the subject. In some embodiments, the test result may comprise the amount of activated HER3 in a tumor sample associated with the subject. In some embodiments, the processor 1002 may determine the test result based at least in part on the received laboratory test results for the subject, the test results comprising the amount of total HER3 and at least one of the amount of HER2/HER3 heterodimers, phosphorylated HER3, or HER3/PI3K complex, and may also include the ratio of at least one of HER2-HER3 heterodimer, phosphorylated HER3, or HER3/PI3K complex to total HER3 protein in a tumor sample obtained from the subject. In some embodiments, the processor 1002 may further determine information about the amount of activated HER3, as measured by the ratio of at least one of HER2-HER3 heterodimer, phosphorylated HER3, or HER3/PI3K complex to total HER3 protein, in a reference population. For example, in some embodiments, the processor 1002 may determine the amount of total HER3 and the ratio of at least one of HER2-HER3 heterodimer, phosphorylated HER3, or HER3/PI3K complex to total HER3 protein measured in a tumor sample obtained from the subject. In some embodiments, the processor 1002 may also determine the ratio of at least one of HER2-HER3 heterodimer, phosphorylated HER3, or HER3/PI3K complex to total HER3 protein measured in a tumor sample obtained from the subject. In some embodiments, the processor 1002 may compare the amount of total HER3 and the ratio of at least one of HER2-HER3 heterodimer, phosphorylated HER3, or HER3/PI3K complex in the tumor sample obtained from the subject to total HER3 protein measured in a tumor sample obtained from the subject are compared to the the median amount of HER3 and the median ratio of at least one of HER2-HER3 heterodimer, phosphorylated HER3, or HER3/PI3K complex to total HER3 protein in the reference population of subjects having the same type of cancer as the subject.

The method 1100 continues at step 1112 when processor 1002 generates a test result report for the subject, the test result report comprising the amount of activated HER3 in the tumor sample. The test result report may comprise the additional determined information about the amount of activated HER3. In some embodiments, the test result report may comprise the amount of activated HER3 in the tumor sample and be based at least in part on the test result for the subject. For example, in some embodiments, the test report may include the amount of total HER3 and at least one of HER2-HER3 heterodimer, phosphorylated HER3, or HER3/PI3K complex measured in a tumor sample obtained from the subject, the ratio of at least one of HER2-HER3 heterodimer, phosphorylated HER3, or HER3/PI3K complex to total HER3 protein measured in a tumor sample obtained from the subject, and/or the median amount of HER3 and the median ratio of at least one of HER2-HER3 heterodimer, phosphorylated HER3, or HER3/PI3K complex to total HER3 protein in the reference population of subjects having the same type of cancer as the subject. In some embodiments, the test result report comprises information that the tumor sample from the subject has a high amount of activated HER3 if (i) the amount of total HER3 in the sample is above the median amount of total HER3 of a reference population and (ii) the ratio of at least one of HER2-HER3 heterodimer to HER3 total, phosphorylated HER3 to total HER3 in the sample, or HER3/PI3K complex is above the median ratio of at least one of HER2-HER3 heterodimer to HER3 total, phosphorylated HER3 to total HER3, or or HER3/PI3K complex to total HER3 in the reference population. In some embodiments, the test result report comprises information that the subject is more likely to respond to the HER3-targeted therapy if the tumor sample from the subject is characterized as having a high level of activated HER3, wherein a high level of activated HER3 comprises (i) the amount of total HER3 in the tumor sample from the subject being above the median amount of total HER3 of a reference population of subjects having the same type of cancer as the subject and (ii) the ratio of at least one of the amount of HER2-HER3 homodimer to the amount of HER3 total, the amount of phosphorylated HER1 to the amount of total HER3, or the amount of HER3-PI3K complex to the amount of HER3 total in the tumor sample being above the median ratio of at least one of the amount of HER2-HER3 homodimer to the amount of HER3 total, the amount of phosphorylated HER3 to the amount of total HER3, or the amount of HER3-PI3K complex to the amount of HER3 total, in the reference population of subjects having the same type of cancer as the subject. In some embodiments, the test result report may include information about HER3-targeted drugs.

In some embodiments, test result report may further comprise the amount of total HER2 protein in measured in the sample from the subject. The test result report may then comprises information of whether the subject has a HER2 positive cancer or a HER2 negative cancer. In some embodiments, the HER2 negative cancers and HER2 positive cancers have been characterized by immunohistochemical or in situ hybridization analysis (e.g., at a centralized testing laboratory). In some embodiments, the test result report may further comprise information that a co-therapy comprising a HER2-targeted therapy and a HER3-targeted therapy would be an appropriate therapy if the subject has a HER2 positive cancer. In some embodiments, the test result report may further comprise information indicating that the subject is more likely to respond to a co-therapy of a HER2-targeted agent and a HER3-targeted agent if the subject has a HER2 positive cancer. In certain embodiments, the test result report may further comprise information of whether the amount of total HER2 protein in the subject sample is below a first cutoff comprising a level of total HER2 protein corresponding to the bottom 5^{th} percentile of total HER2 protein expression in a reference population of HER2 positive cancers (i.e., HERmark® HER2 negative), if the amount of total HER2 protein is above a second cutoff comprising a level of total HER2 protein corresponding to a top 95^{th} percentile of total HER2 protein expression in a reference population of HER2 negative cancers (i.e., HERmark® HER2 positive), or whether the amount of total HER2 protein is above the first cutoff but below the second cutoff (i.e., HERmark® HER2 positive). In certain embodiments, the test result report may further comprise information that a co-therapy comprising a HER2-targeted therapy and a HER3-targeted therapy would be an appropriate treatment for the subject if the amount of total HER2 protein in the tumor sample is above the first cutoff. In certain embodiments, the test result report may further comprise information indicating that the subject is more likely to respond to a co-therapy of a HER2-targeted agent and a HER3-targeted agent if the amount of total HER2 protein in the biological sample is above the first cutoff. In some embodiments, the test result report may include information about HER3-targeted drugs and/or HER2-targeted drugs.

The method 1100 continues at step 1114 when processor 1002 stores the test result report for the subject in the database 1018. The processor 1002 may transmit data associated with the test result report to the database 1018. In some embodiments, a healthcare provider (e.g., the patient's healthcare provider) may be able to access the stored test result report. For example, in some embodiments, the healthcare provider may be able to query the database 1018 directly or indirectly.

The method 1100 continues at step 1116 when processor 1002 provides an electronic notification to a computing device 1028. The electronic notification may indicate that a test report is available, for example, for download from the database 1018. In some embodiments, the computing device 1028 may be associated with a healthcare provider 1026 or the subject. In some embodiments, the electronic notification may comprise an e-mail, a text message, or a push notification.

The method 1100 continues at step 1118 when processor 1002 transmits the test result report for the subject to a computing device 1020. In some embodiments, the processor 1002 may transmit the test result report to a computing device 1020 associated with the subject. In some embodiments, the processor 1002 may transmit the test result report to a computing device 1026 associated with a healthcare provider. In some embodiments, the processor 1002 may transmit the test result report via e-mail (e.g., to an e-mail account associated with the subject's healthcare provider), SMS, or text message.

It should be understood that the foregoing relates to certain embodiments of the invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein may suggest themselves to those skilled in the art without departing from the spirit of the present invention and/or the scope the appended claims.

All printed patents and publications referred to in this application are hereby incorporated herein in their entirety by this reference.

### EXAMPLES

The present invention may be better understood by reference to the following non-limiting examples.

### Example 1

### Tissue Samples

Fifty-six breast tumor samples were assessed: 38 were purchased from Asterand, Inc. (Detroit, Mich.) and 18 were obtained from AstraZeneca. These samples had been characterized as either HER2-positive or HER2-negative as determined by IHC. The majority of samples are ductal carcinoma. The HERmark® assay was used to measure total HER2 protein in these samples and they were characterized as HERmark® HER2 positive (n=24), HERmark® HER2 negative (n=27), and HERmark® equivocal (n=5), using the cutoffs described in Example 7.

### Example 2

### Fixation, Processing and Paraffin Embedding

All tissues (0.3-1.0 gm) were fixed or snap-frozen within 15-30 minutes of excision. All tissues were fixed identically in neutral-buffered formalin (10% NBF) as dictated by the vendor's Standard Operating Procedure, which is consistent with the ASCO/CAP guidelines for preparation of breast tumor tissue for HER2 testing and other methods for HER3 fixation (i.e., 10% NBF for approximately 24 hrs at 4° C.). An independent study has shown preservation of phosphorylated HER expressed in xenograft tumors collected and processed in a similar manner (Mukherjee et al, 2011).

All cell-lines were maintained at 37° C. and 5% CO₂ in Dulbecco's modified Eagle medium (DMEM): F12 (50:50), 10% FBS, 1% PSQ (10% fetal bovine serum, 1% penicillin-streptomycin) and 2 mM L-glutamine. Cells were grown to near confluence on at least ten 150-mm culture plates for each cell line. After removal of medium, the cells were washed once with cold 1×PBS and 15 mL of 10% NBF (neutral buffered formalin) was added to each plate. Cells were fixed overnight (>16 hrs) at 4° C. After removal of the fixative solution, the cells were harvested by scraping with residual fixative solution and centrifuged at 3200×g for 15 min. The cell pellet was transferred to a rubber O-ring, wrapped with filter paper and placed in a processing cassette. An automatic Tissue-Tek processor was used for dehydration and paraffin infusion processing. Briefly, cell pellet was exposed to increasing concentrations of alcohol, Clear-Rite clearing agent (xylene substitute) and paraffin. After processing, the cell pellet was embedded in a block using a paraffin embedding station. All solvents used for cell pellet processing were obtained from Richard-Allen Scientific.

### Example 3

### Microtomy

Sections of 5 μm in thickness were sliced with a microtome (LEICA) and placed on positively charged glass slides (VWR) with serial number labeled. Slides were air-dried for 30 minutes and then baked in a heated oven set at 60° C. for 1 hour. All sample slides were stored at 4° C. for future assays. For each sample, one slide was stained with hematoxylin and eosin (H&E) and examined by a pathologist for tumor content. Non-tumor elements were identified and macrodissected away to provide tumor enrichment of ≧70%.

### Example 4

### Antibody Conjugate Reagents

Monoclonal antibodies against the intracellular domain of HER2, the intracellular domain of HER3, and phosphorylated tyrosine 1289 of HER3 were used. Primary antibodies against HER2 and HER3, and other reagents, were purchased from Labvision (HER2 cat. #: MS-325 and MS-599, HER3 cat. #: MS-310); Cell Signaling (HER2 cat. #: 2165; phospho-HER3pY1289 cat #4791); Millipore (PI3K (Ab6) cat #05-212); and Southern Biotech (Goat anti-Mouse IgG cat. #: 1030-01, Goat F(ab′)2 anti-Rabbit IgG cat. #: 4052-01). Monoclonal antibody B9A11 is a proprietary monoclonal antibody raised against HER3 by Monogram Biosciences (CA) (ATCC # PTA-10574).

VeraTag® reporter molecules (Proll and Pro125) and streptavidin-conjugated methylene blue (“molecular scissors”) were synthesized and purified according to protocols described, for example, above and in U.S. Pat. Nos. 7,105,308 and 7,255,999, which are incorporated by reference in their entirety herein. VeraTag® reporter molecule-conjugated antibody and biotin-conjugated antibody were made using sulfo-NHS-LC-LC-biotin (Pierce) as linker according to manufacturer's protocol and conjugation products purified by HPLC (Agilent). In certain experiments, the VeraTag® reporter molecule or biotin was conjugated to a primary antibody. Depending on the assay format, the VeraTag® reporter molecule or biotin was conjugated to a secondary antibody that binds to the primary antibody.

In the experiments described below, VeraTag® reporter molecule Proll was used in assays to measure HER2, HER3, HER2-HER3 heterodimers, HER3pY1289, and HER3/PI3K complexes. The assay format for detection of HER2-HER3 heterodimers used an unconjugated primary anti-HER3 mouse monoclonal antibody (B9A11) and unconjugated primary anti-HER2 rabbit monoclonal primary antibody. Target binding by these primary antibodies was then detected through binding of a secondary antibody Goat anti-Mouse IgG conjugated to VeraTag® reporter molelecule Proll and a secondary Goat F(ab′)2 anti-Rabbit IgG conjugated to biotin, respectively. The assay format for detection of HER3/PI3K complexes involved conjugating biotin to the anti-HER3 antibody B9A11, and the VeraTag® reporter molecule Proll to the p85-PI3K antibody (Ab6). No secondary antibodies were used in the HER3/PI3K complex assay format.

### Example 5

### VeraTag® Assay

Various VeraTag® assay formats are shown in FIG. 1. The assay format can be modified to use formalin-fixed paraffin-embedded (FFPE) tissue samples (Panel A) or tissue lysate samples (Panel B). In addition, the VeraTag® reporter molecules may be cleaved from antibodies to which they are bound using either a light release format (Panels A and B) or using a reducing format, e.g., using DTT (Panel C). In the light release format, diffusing reactive singlet oxygen cleaves the covalent linker between a VeraTag® reporter molecule and a HER3 antibody, for example, in response to photo-induction of the cleavage-inducing agent by light (“hv”).. In the reducing format, a reducing agent such as, e.g., DTT, may be used to cleave the covalent linker between a VeraTag® reporter molecule and a HER3 antibody. Following cleavage, capillary electrophoretic (CE) separation may be performed to separate out the cleaved VeraTag® reporter molecules (tags), which can be visualized in an electropherogram. The x-axis of the resulting electropherogram shows the time at which the cleaved VeraTag® reporter molecule eluted from the capillary (i.e., based on electrophoretic mobility), and the fluorescence intensity is shown on the y-axis. Where the VeraTag® assay is formatted to detect the amount or presence of more than one protein target, target specific antibodies may be conjugated to VeraTag® reporter molecules having different electrophoretic mobilities so that each tag may be identified and quantified on the electropherogram.

The following method describes a general VeraTag® light-release assay that can be used to measure biomarker levels in a biological sample.

Deparaffinization and antigen retrieval was generally performed as in (Sperinde et al, 2010, Clin. Cancer Res. 16(16):4226-4235; Shi, Y., et al., 2009, Diagn. Mol. Pathol. 18(1):11-21). FFPE samples were deparaffinized/rehydrated using a series of solvents. Briefly, slides were sequentially soaked in xylene (2×, 5 min), 100% ethanol (2×, 5 min), 70% ethanol (2×, 5 min) and deionized water (2×, 5 min). Heat-induced epitope retrieval of the rehydrated samples was performed in a dish containing 250 mL of 1× citrate buffer (pH 6.0) (Lab Vision) using microwave oven (Spacemaker II, GE): 3 min at power 10 followed by 10 min at power 3. After being cooled down for 20 min at room temperature, the slides were rinsed once with deionized water. A hydrophobic circle was drawn around the section on the slide using a hydrophobic pen (Zymed) to retain reagents on slides. The samples were then blocked for 1 hour with blocking buffer that contains 1% mouse serum, 1.5% BSA and a cocktail of protease and phosphatase inhibitors (Roche) in 1×PBS.

After removal of the blocking buffer with aspiration, a mixture of VeraTag® reporter molecule- and biotin-conjugated antibodies prepared in blocking buffer was added, and binding reactions were incubated overnight in a humidified chamber at 4° C. with shaking. The antibody mix was aspirated and samples were washed with buffer containing 0.25% Triton X-100 in 1×PBS, and streptavidin-conjugated methylene blue at a concentration of 2.5 μg/mL in 1×PBS was added. The concentrations of the antibody and streptavidin-photosensitizer conjugates were all optimized based on signal specificity and assay dynamic range using both cell line and breast tissue samples. After 1 hour incubation at room temperature, the streptavidin-methylene blue reagent was aspirated and the samples were washed in wash buffer once followed by 3 changes of deionized water. Illumination buffer containing 3 pM fluorescein and two CE internal markers (MF and ML) in 0.01×PBS was added on sample sections. The bound VeraTag® reporter molecule was released at ˜4° C. by photo-activated cleavage using an in-house LED array illuminator equipped with an electronic ice cube/chiller block (Torrey Pine Scientific). The CE sample containing the released VeraTag® reporter molecules was collected from above the tissue section on the slides and the released VeraTag® reporter molecules in the CE samples were separated and detected on an ABI3100 CE instrument (22-cm capillary array) (Applied Biosystems) under CE injection condition of 6 kV and 50 sec at 30° C.

The reducing (DTT) release assay format is similar but with the following general differences. After removal of the Blocking Buffer with aspiration, a solution containing the biomarker-specific antibody (e.g., HER3 antibody) in Blocking Buffer was added to the slides and left at 4° C. overnight in a humidified chamber with gentle shaking. The antibody solution was aspirated and samples were washed with PBS containing 0.25% TritonX-100 for 5 minutes then PBS alone for 5 minutes. Following aspiration, secondary antibody labeled with a VeraTag® reporter molecule in Blocking Buffer was added. The secondary antibody was allowed to incubate at room temperature for 1.5 hours in a humidified chamber. The slides were next rinsed with deionized water followed by PBS containing 0.25% Triton X-100 for 5 minutes. Slides were then loaded onto racks and submerged in deionized water 6 times. Following centrifugation of the slides, 100 μL Capture Buffer containing 1.0 mM dithiothreitol (DTT), 3 pM fluorescein and two CE internal markers (MF and ML) in 0.01×PBS was added on sample sections. Slides were incubated in a humidified chamber for 2 hours to allow for the release of the VeraTag® reporter molecule. Capture Buffer from each slide was transferred to a CE 96-well plate then diluted appropriately (generally 10-fold) in Capture Buffer not containing DTT. The released VeraTag® reporter molecules in the CE samples were separated and detected on a ABI3100 CE instrument (22-cm capillary array, Applied Biosystems) under the CE injection condition of 6 kV and 50 sec.

The biomarkers as described herein may be detected using a VeraTag® assay similar to as described above but using protein specific antibodies. These assays are identified in Table 1 below. Each of these VeraTag® assays used a blocking buffer of 10% goat serum (Sigma), 1 mg/ml hIgG (Sigma), 1.5% BSA and a cocktail of protease and phosphatase inhibitors (Roche) in 1×PBS. The concentrations of VeraTag® reporter molecule- and biotin-conjugated antibodies used for these assays are also indicated in Table 1.

TABLE 1
VeraTag ® Assay Formats

| Biomarker | Assay Conditions |
|---|---|
| HER2 Total | Release Method: light release method |
| | Antibody Format: two different HER2 antibodies |
| | Antibody Concentrations: both antibodies used at 4 μg/mL |
| | Heat Retrieval Buffer: Diva Decloaker buffer, pH 6.2 |
| HER3 Total | Release Method: light release method |
| | Antibody Format: two different HER3 antibodies |
| | Antibody Concentrations: both antibodies used at 2 μg/mL |
| | Heat Retrieval Buffer: DAKO Target Retrieval Solution, pH 9 (Dako |
| | Corporation) |
| HER3pY1289 | Release Method: light release method |
| | Antibody Format: single HER3 antibody and single HER3pY1289 |
| | specific antibody |
| | Antibody Concentrations: both antibodies used at 2 μg/mL |
| | Heat Retrieval Buffer: Borg Decloaker buffer, pH 9.5 (Biocare Medical) |
| HER2-HER3 | Release Method: light release method |
| heterodimer | Antibody Format: single HER3 antibody, single HER2 antibody, first |
| | secondary antibody for HER3 antibody, second secondary antibody for |
| | HER2 antibody |
| | Antibody Concentration: HER2 primary antibody at 1 μg/mL and HER2 |
| | secondary conjugated to biotin at 0.5 μg/mL; HER3 primary antibody at |
| | 0.25 μg/mL and HER3 secondary conjugated to VeraTag ® reporter |
| | molecule at 0.5 μg/mL |
| | Heat Retrieval Buffer: DAKO Target Retrieval Solution, pH 9 (Dako |
| | Corporation) |
| HER3/PI3K | Release Method: light release method |
| complex | Antibody Format: single HER3 antibody, single p85-PI3K antibody |
| | Antibody Concentration: HER3 primary antibody (B9A11) conjugated |
| | to biotin at 3 μg/mL; PI3K primary antibody conjugated to VeraTag ® |
| | reporter molecule at 0.5 μg/mL |
| | Heat Retrieval Buffer: DAKO Target Retrieval Solution, pH 9 (Dako |
| | Corporation) |

The readout for the different formats of the VeraTag® assay is RPA×IB×BNF/TA. This readout is defined as follows: RPA=Relative Peak Area of the fluorescence of the released VeraTag, purified and quantified by capillary electrophoresis relative to an internal standard that corrects for recovery (e.g., fluorescein peak area); IB=the volume of the Illumination Buffer used during the photoactivation and release of the fluorescent tag; BNF=Batch Normalization Factor obtained from the control cell lines assayed in every batch and used to normalize for batch to batch variation in signal; TA=Tumor Area of the sample determined by post-assay staining by H&E stain, and measurement by a licensed pathologist. This is used to normalize the fluorescent assay signal to amount of tumor.

### Example 6

### VeraTag® Assay Batch Consistency

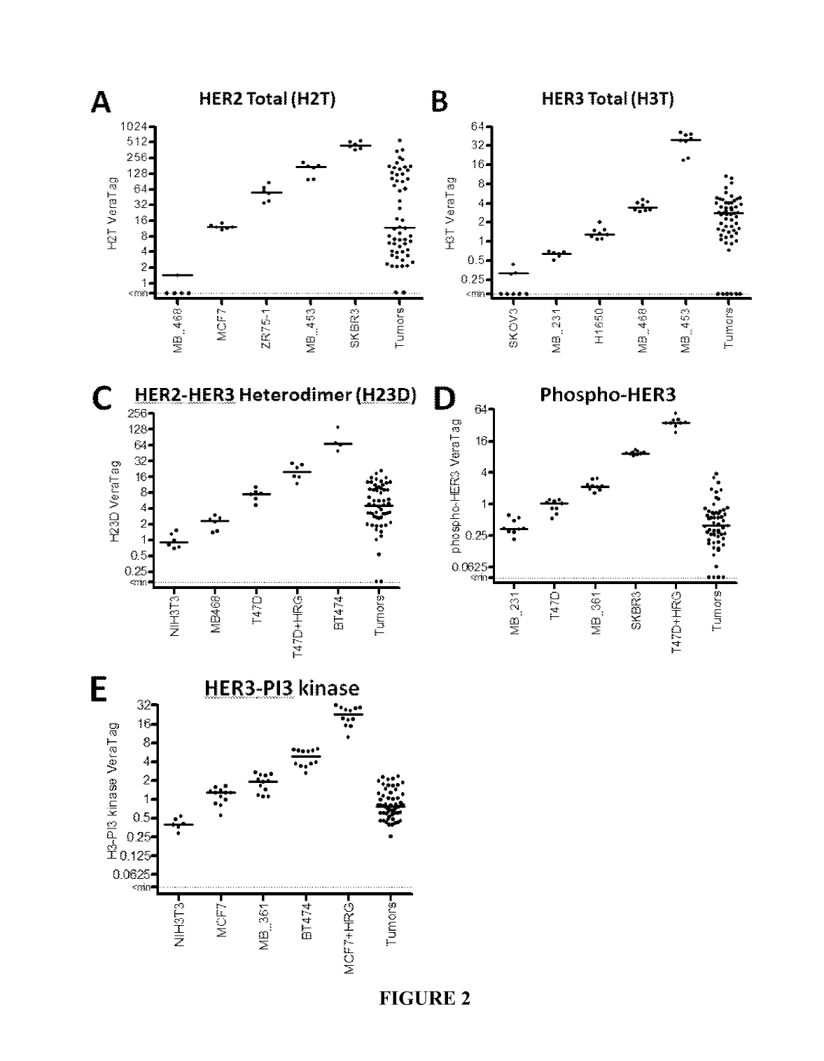

Due to the number of tumor samples assessed, VeraTag® assays were run for batches of tumor samples. The cell lines controls were also assessed for each batch to normalize measurements between batches. Scatter plots of biomarker measurements of the tumor samples are show in FIG. 2. The fluorescence intensity for each sample is shown on the y-axis, measured in relative peak area (RPA). Control cell line measurements are on the left side of each graph and tumor sample measurements are on the right side of each graph. The solid dots indicate signal, and the open dots represent background.

Panels A and B of FIG. 2 show results from assays measuring levels of total HER2 and HER3, respectively. Panels C, D, and E of FIG. 2 show results from assays measuring activated HER3; specifically, HER2-HER3 heterodimers, HER3 phosphorylated at tyrosine 1289 (Phospo-HER3), and HER3/PI3K complexes, respectively.

The measurements for the cell line controls were found to be consistent from batch to batch, indicating the analytical reproducibility from batch to batch for the VeraTag® assay measurements.

### Example 7

### Characterization of Tumors Using HERmark® HER2 Assay

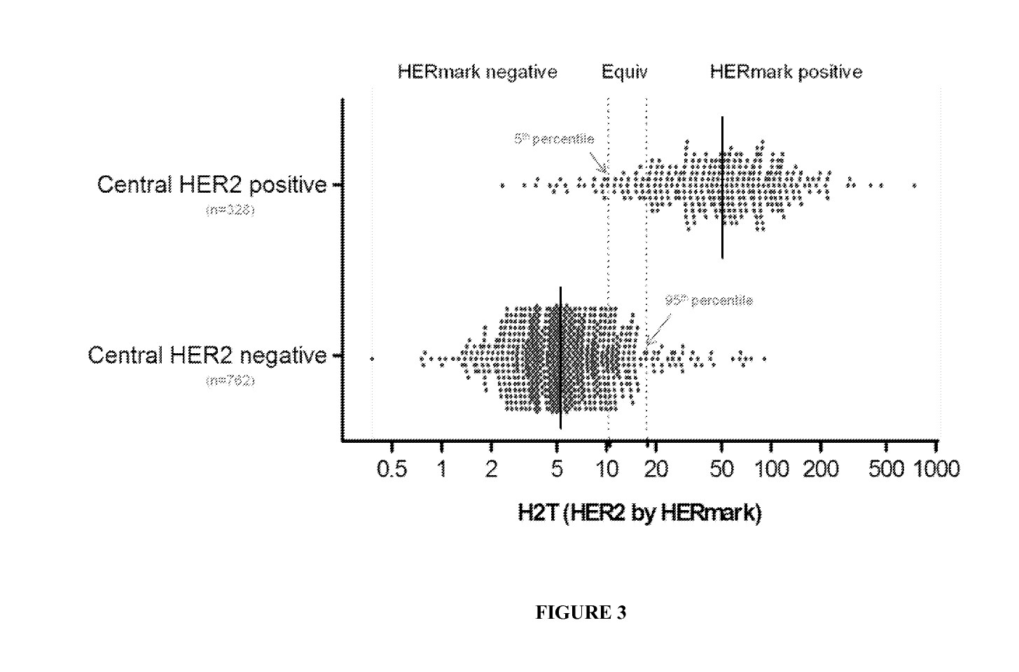

Cutoffs were previously established for the HERmark® assay in characterizing tumor samples based on total HER2 levels according to methods described in Huang et al., Am. J. Clin. Pathol. 134:303-311 (2010). Total HER2 expression was determined using the HERmark® assay (Monogram Biosciences, CA) for 1,090 breast cancer samples that had been previously characterized at a central testing facility as either HER2 positive or HER2 negative by current clinical standard guidelines using immunohistochemistry (IHC) and/or in situ hybridization (ISH) (i.e., FISH positive, IHC 3+ score, or IHC 2+ and FISH+). This data is shown as a scatter plot in FIG. 3. The cutoff for HERmark® HER2 positive was set as expression greater than (above) the 95th percentile of HER2 expression of the samples classified as HER2 negative by reference methods (IHC0, IHC1+ or IHC2+/ISH− or ISH−). The cutoff for HERmark® HER2 negative was set as expression less than (below) the 5^{th} percentile of the samples classified as HER2 positive by reference methods (IHC3+ or IHC2+/ISH+ or ISH+). HERmark® HER2 equivocal status was set as expression levels falling in the overlap between the 5^{th} percentile cutoff for the HER2-positive samples and the 95^{th} percentile cutoff for the HER2 negative samples (i.e., between the two cutoffs).

### Example 8

### Biomarker Levels Differ Between HERmark® HER2 Positive and HERmark® HER2 Negative Breast Cancers

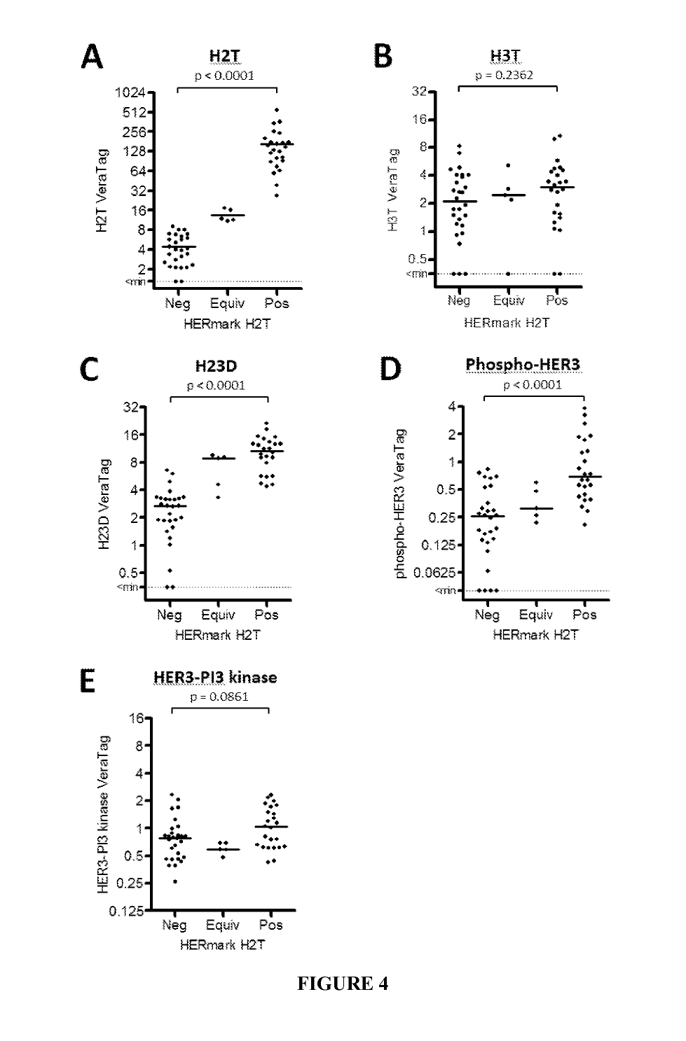

Graphs showing the amounts of different biomarkers in the breast tumor samples are shown in FIG. 4. The biomarkers assessed were: total HER2 (H2T), total HER3 (H3T), HER2-HER3 heterodimers (H23D), HER3 phosphorylated at tyrosine 1289 (Phospho-HER3), and HER3/PI3K complexes (HER3-PI3 kinase). See FIG. 4, Panels A-E, respectively. Biomarker levels were measured using VeraTag® assays as described in Example 5. Biomarker levels were plotted using the scatter method. Sample numbers are indicated on the x axis, and VeraTag® assay readout (RPA×IB×BNF/TA) is shown on the y-axis. A Mann-Whitney statistical analysis was performed to determine if the mean biomarker levels were different between the HERmark® HER2 negative cancers and the HERmark® HER2 positive cancers. While the distribution and median level of HER3 is similar in high and low HER2 breast tumors, the distribution and median level of activated HER3 measured as HER2-HER3 heterodimers and phosphorylated HER3 is significantly greater in high HER2 breast tumors when compared to the low HER2 tumors, supporting a major role for HER2 in HER3 signaling.

### Example 9

### Biomarker Level Correlations and Spearman Correlation Coefficient Analysis

Statistical analysis of the biomarkers levels was conducted to determine if the levels of each biomarker was correlated to the level of any other biomarker in the tumor samples. Biomarker levels were measured using VeraTag® assays as described in Example 5. Spearman's Rank Correlation test analysis was performed to identify a Spearman's rank correlation coefficient (Spearman) and p-value for each pairwise comparison of biomarkers.

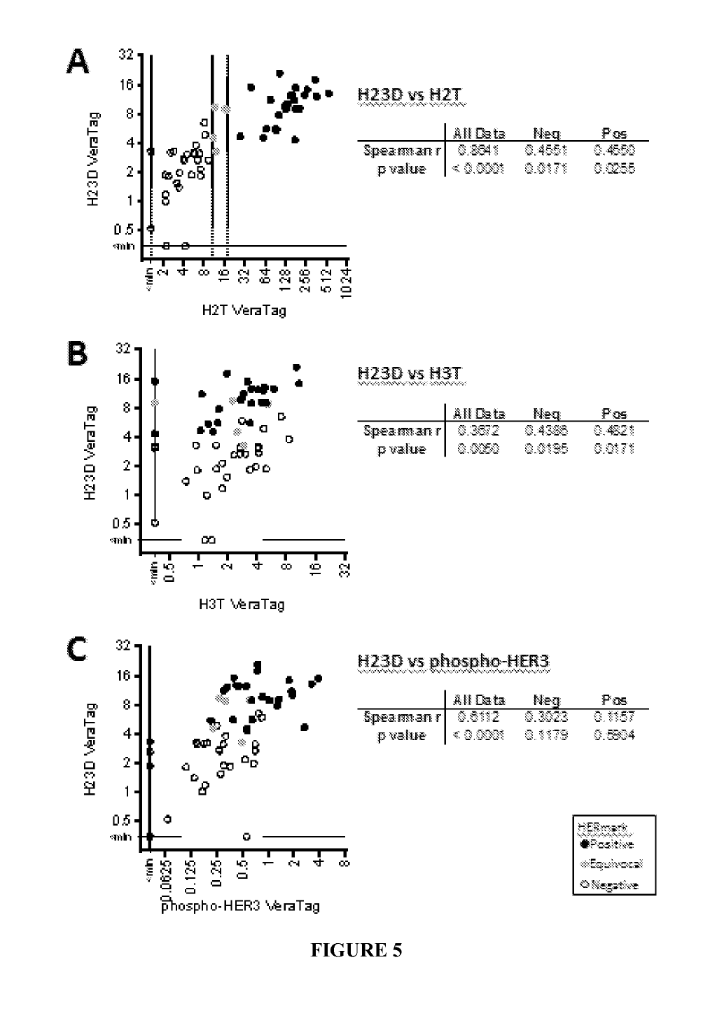

Graphs illustrating the statistical relationship levels between the markers measured in FIG. 3 (high versus low HER2 total) and activated HER3 are shown in FIG. 5. Panel A shows the correlation between HER2-HER3 heterodimers and total HER2. Panel B shows the correlation between HER2-HER3 heterodimers and total HER3. Panel C shows the correlation between HER2-HER3 heterodimers and phosphorylated HER3. Although there is a significant correlation between HER2-HER3 heterodimers and HER2 total, and between HER2-HER3 heterodimers and HER3 total, the correlation is greater with HER2 total (Spearman r=0.8641 vs 0.3572), supporting a major role of HER2 in HER3 signaling. The significant correlation overall between HER2-HER3 heterodimers and phosphorylated HER3 indicates that activated HER3 is consistent in a given sample as measured by two different methods.

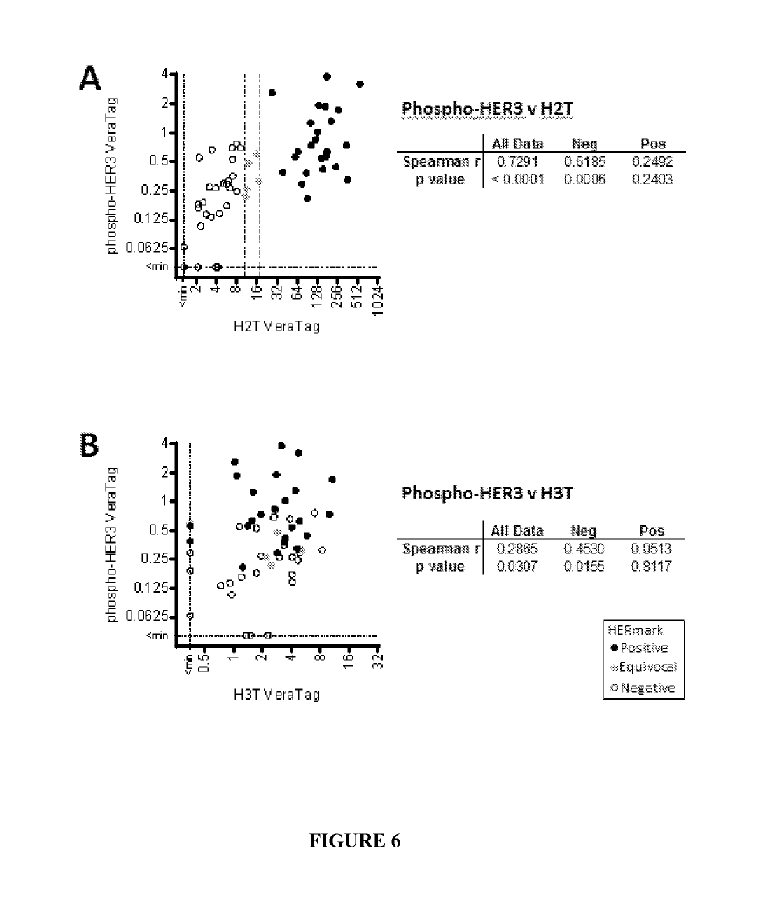

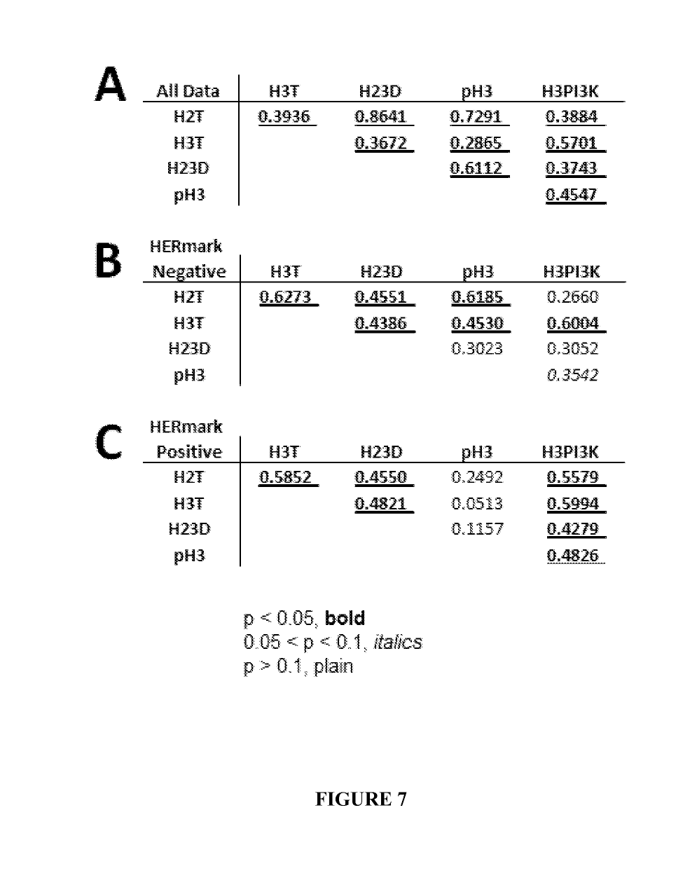

Graphs illustrating the statistical relationship between the markers measured in FIG. 3 (high versus low HER2 total) and phosphorylated HER3 are shown in FIG. 6. Panel A shows the correlation between phosphorylated HER3 and HER2 total. Panel B shows the correlation between phosphorylated HER3 and HER3 total. In both cases the overall correlation between phosphorylated HER3 and HER2 total, and phosphorylated HER3 and HER3 total is significant, however, the activation of HER3 measured by phosphorylated HER3 is more tightly correlated with HER2 total than with HER3 total (Spearman r=0.7291; p<0.0001 vs 0.2865; p<0.0307). Tables summarizing the results of this statistical analysis are shown in FIG. 7 (Panel A: All samples; Panel B: HERmark® HER2 negative samples; Panel C: HERmark® HER2 positive samples). The Spearman's rank correlation coefficients having significant p-values (p<0.05) are underlined.

### Example 10

### Heat Map Illustrating Correlation of Marker Levels

Biomarker levels were measured using VeraTag® assays and plotted onto heat maps to determine if a subset of samples for each cancer could be identified as having highly activated HER3. As HER3-targeted therapies are more likely to be effective to treat cancers that are significantly driven by HER3 activation, identifying subjects with HER3-activated cancers may help to identify subjects who will respond better to HER3-targeted therapies. The levels for different biomarkers, as measured by VeraTag® assays, were plotted onto heat maps to identify expression patterns.

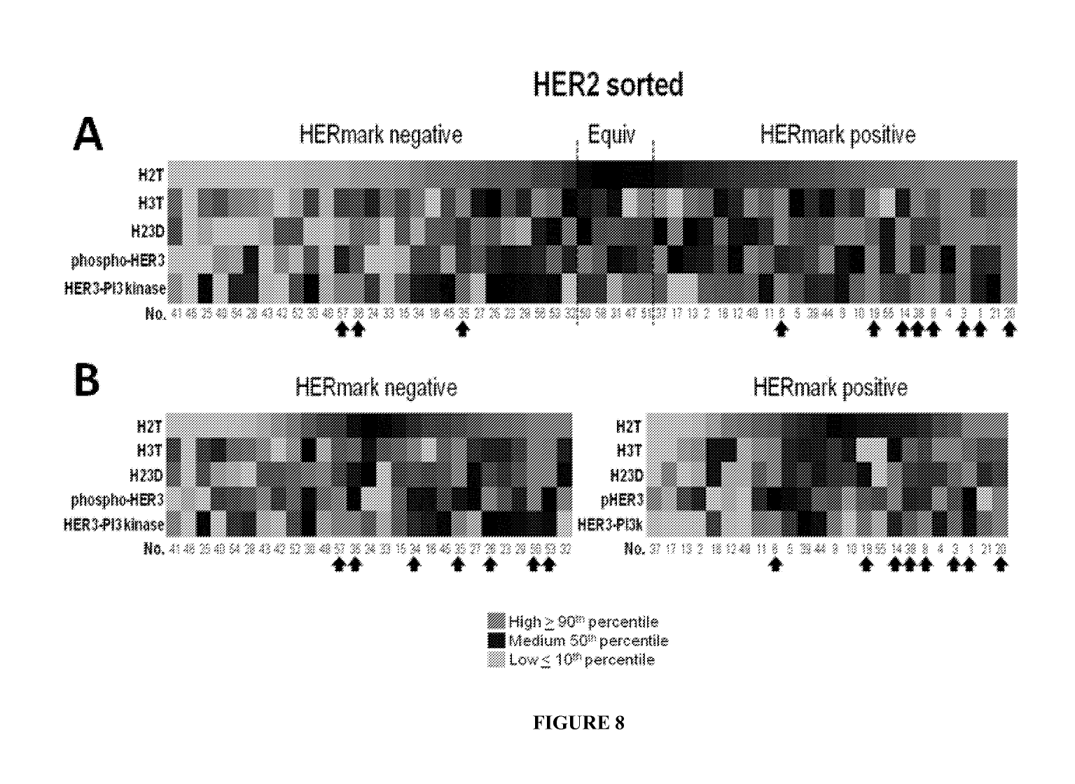

FIG. 8 shows the heat map for the samples. Panel A is a heat map showing biomarker levels for all breast cancer tumor samples sorted from high to low HERmark® HER2 status. Panel B shows two heat maps in which the tumor samples have been segregated based on HERmark® HER2 status.

The sample number for each tumor sample is indicated at the bottom of each heat map, and the biomarker analyzed in the assay is shown to the left of each heat map. Samples that exhibited the highest expression (≧90^{th} percentile) are shown in dark grey; samples with medium expression (50^{th} percentile) are shown in black, and samples with low expression (≧10^{th} percentile) are shown in light grey. Intermediate shading reflects intermediate expression levels. Samples having the highest levels of activated HER3 are marked with an arrow. Samples were categorized as having the highest levels of activated HER3 based on evaluation of moderate to high HER3 total measurements combined with high levels of at least one of HER2-HER3 heterodimers, HER3 phosphorylation, and HER3/PI3K complex (i.e., recruitment of PI3K to activated HER3).

For example, sample 6 in FIG. 8A has moderate levels of HER2 total but very high levels of HER2-HER3 heterodimers. As dimerization is necessary for receptor activation, very high levels of dimerization are likely to indicate increased HER3 receptor activation. Another example is sample 19 in FIG. 8A, which had moderate levels of HER2 total but very high levels of HER3phospho-1289 levels. As phosphorylation of HER3 at position 1289 activates HER3 signaling, high levels of this biomarker were also considered indicative of increased HER3 activation in a sample. Heat maps profiling the HER3 pathway allowed for the identification of HER3 activated samples not seen by stratification of HER2 status alone. Heat maps ranked by HER2 total identified a subset of samples that grouped following cluster analysis (Example 11). Phospho-HER3 and HER3-PI3 kinase complexes significantly correlate with HER2 total levels overall (FIG. 7) but high levels of phospho-HER3 and HER3-PI3 kinase complexes in a subgroup of HERmark® HER2 low breast cancer samples suggest there may also be additional mechanisms of HER3 activation besides amplified HER2.

### Example 11

### Hierarchical Cluster Analysis of Breast Cancer Samples

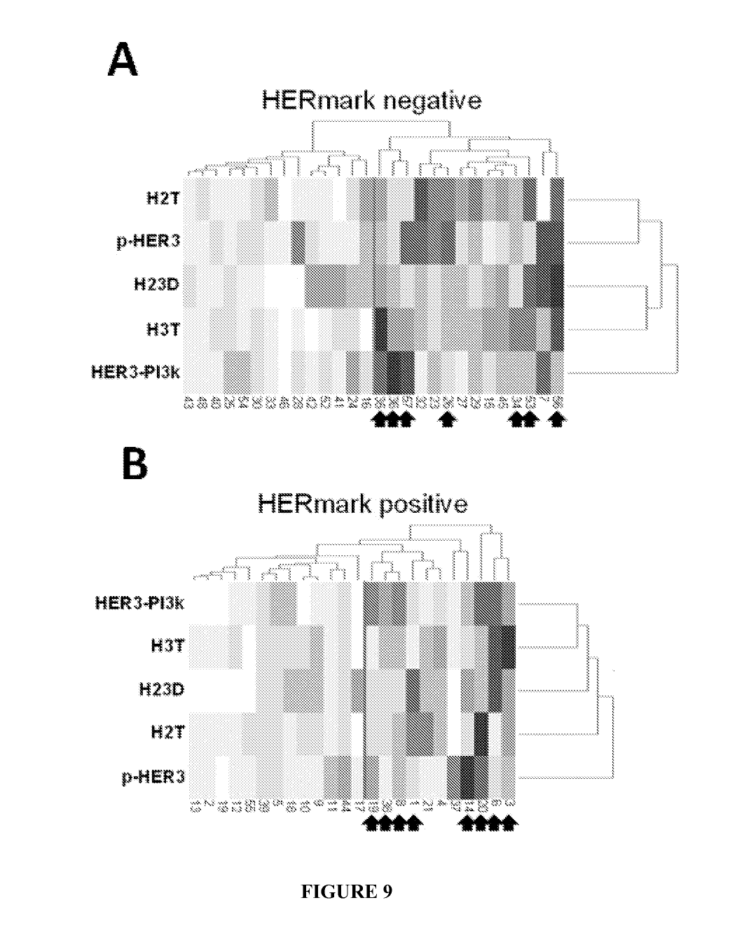

FIG. 9 shows a hierarchical cluster analysis of the tumor samples by biomarker level as measured by VeraTag® assays. Panel A shows the analysis for HERmark® HER2 negative breast cancer samples, and Panel B shows the analysis for HERmark® HER2 positive breast cancer samples. The sample numbers are shown on the bottom of the graphs. The biomarkers analyzed are shown along the left side of the graph: HER3/PI3K complex (HER3-PI3K), total HER3 (H3T), HER2-HER3 heterodimers (H23D), total HER2 (H2T), and HER3 phosphorylated at tyrosine 1289 (p-HER3). The tumors expressing the highest levels of activated HER3 cluster into the right side of the graphs. The resulting dendogram identified subgroups of tumors with activated HER3 that are the same as those identified by the H2T-ranked heat maps.

All printed patents and publications referred to in this application are hereby incorporated herein in their entirety by this reference.

While the preferred embodiment of the invention has been illustrated and described, it will be appreciated that various changes can be made therein without departing from the spirit and scope of the invention.

That which is claimed is:

1. A method for measuring the amount of activated HER3 in a tumor, comprising:
(a) measuring in a tumor sample the amount of total HER3 and the amount of at least one of HER2-HER3 heterodimer, phosphorylated HER3, or HER3/PI3K complex in the sample;
(b) determining the ratio of at least one of HER2-HER3 heterodimer, phosphorylated HER3, or HER3/PI3K complex to total HER3 protein; and
(c) indicating that the tumor has a high amount of activated HER3 if (i) the amount of total HER3 in the sample is above the median amount of total HER3 of a reference population and (ii) the ratio of at least one of HER2-HER3 heterodimer to HER3 total, phosphorylated HER3 to total HER3 in the sample, or HER3/PI3K complex is above the median ratio of at least one of HER2-HER3 heterodimer to HER3 total, phosphorylated HER3 to total HER3, or or HER3/PI3K complex to total HER3 in the reference population.

2. A method of treating a subject with cancer comprising:
(a) measuring in a tumor sample from the subject the amount of total HER3 protein and at least one of HER2-HER3 heterodimer, phosphorylated HER3, or HER3/PI3K complex;
(b) determining the ratio of the amount of at least one of HER2-HER3 heterodimer, phosphorylated HER3, or HER3/PI3K complex to total HER3;
(c) determining if a subject has a cancer characterized as having a high level of activated HER3, wherein a high level of activated HER3 comprises (i) the amount of total HER3 in the sample being above the median amount of total HER3 of a reference population of subjects having the same type of cancer as the subject and (ii) the ratio of at least one of the amount of HER2-HER3 homodimer to the amount of HER3 total, the amount of phosphorylated HER1 to the amount of total HER3, or the amount of HER3-PI3K complex to the amount of HER3 total in the sample being above the median ratio of at least one of the amount of HER2-HER3 homodimer to the amount of HER3 total, the amount of phosphorylated HER3 to the amount of total HER3, or the amount of HER3-PI3K complex to the amount of HER3 total, in the reference population of subjects having the same type of cancer as the subject; and
(d) administering a HER3-targeted therapy to the subject if the subject has a cancer characterized as having a high level of activated HER3.

3. A method for predicting responsiveness of a subject with cancer to a HER3-targeted therapy comprising:
(a) measuring in a biological sample from the subject's cncer the amount of total HER3 protein and at least one of HER2-HER3 heterodimer, phosphorylated HER3, or HER3/PI3K complex;
(b) determining the ratio of the amount of at least one of HER2-HER3 heterodimer, phosphorylated HER3, or HER3/PI3K complex to total HER3;
(c) determining if a subject has a cancer characterized as having a high level of activated HER3, wherein a high level of activated HER3 comprises (i) the amount of total HER3 in the sample being above the median amount of total HER3 of a reference population of subjects having the same type of cancer as the subject and (ii) the ratio of at least one of the amount of HER2-HER3 homodimer to the amount of HER3 total, the amount of phosphorylated HER1 to the amount of total HER3, or the amount of HER3-PI3K complex to the amount of HER3 total in the sample being above the median ratio of at least one of the amount of HER2-HER3 homodimer to the amount of HER3 total, the amount of phosphorylated HER3 to the amount of total HER3, or the amount of HER3-PI3K complex to the amount of HER3 total, in the reference population of subjects having the same type of cancer as the subject; and
(d) indicating that the subject is more likely to respond to the HER3-targeted therapy if the subject's cancer is characterized as having a high level of activated HER3.

4. The method of any one of claims 1-3, wherein the tumor comprises at least one of colorectal cancer, gastric cancer, breast cancer, melanoma, ovarian cancer, head and neck cancer, lung cancer, brain cancer, endometrial cancer, pancreatic cancer, prostate cancer, or cervical cancer.

5. The method of any one of claims 1-3, wherein the tumor comprises breast cancer.

6. The method of any one of claims 1-3, wherein the amount of phosphorylated HER3 in the biological sample is detected by using a HER3 phosphospecific or a HER3 pan-phospo antibody.

7. The method of any one of claims 1-3, wherein the amount of phosphorylated HER3 in the tumor is detected by using a phosphospecific antibody that binds HER3 protein that is phosphorylated at the tyrosine residue at position 1289 of HER3.

8. The method of any one of claims 1-3, wherein the amount of activated HER3 in the tumor is detected by determining at least two of the ratio of the amount of HER2-HER3 heterodimer to the amount of total HER3, the ratio of the amount of phosphorylated HER3 to the amount of total HER3, and the ratio of the amount of HER3/PI3K complex to the amount of total HER3 that is present in the tumor sample.

9. The method of any one of claims 1-3, wherein the amount of HER2-HER3 heterodimer and HER3/PI3K complex are measured using an assay capable of measuring an amount of protein-protein interactions in the tumor sample.

10. The method of any one of claims 1-3, wherein measuring the amount of total HER3 protein in the tumor sample from the subject comprises the steps of:
a) contacting the tumor sample with a HER3 antibody composition;
b) contacting the HER3 antibody composition with a tagged binding composition, wherein the tagged binding composition comprises a molecular tag attached thereto via a cleavable linkage, and wherein the tagged binding composition specifically binds to the HER3 antibody composition;
c) cleaving the cleavable linker of the tagged binding composition, thereby releasing the molecular tag; and
d) quantitating the released molecular tag to determine the amount of HER3 protein in the tumor sample.

11. The method of any one of claims 1-3, wherein measuring the amount of total HER3 protein in the tumor sample from the subject comprises the steps of:
a) contacting the tumor sample with a first HER3 antibody composition that specifically binds to HER3 protein at a first binding site, wherein the first HER3 binding composition comprises a molecular tag attached thereto via a cleavable linkage;
b) contacting the tumor sample with a cleaving probe that specifically binds to HER3 protein at a second binding site, wherein the cleaving probe cleaves the cleavable linkage of the HER3 antibody composition when within an effective proximity thereto;
c) cleaving the cleavable linker of the HER3 antibody composition, thereby releasing the molecular tag; and
d) quantitating the released molecular tag to determine the amount of HER3 protein in the tumor sample.

12. The method of any one of claims 1-3, wherein measuring the amount of HER2-HER3 heterodimer or HER3/PI3K complex in the tumor sample from the subject comprises the steps of:
a) contacting the tumor sample with an antibody composition comprising a molecular tag attached thereto via a cleavable linkage;
b) contacting the tumor sample with a cleaving probe, wherein the cleaving probe cleaves the cleavable linkage of the antibody composition when within an effective proximity thereto;
c) cleaving the cleavable linker of the antibody composition, thereby releasing the molecular tag; and
d) quantitating the released molecular tag to determine the amount of HER2-HER3 heterodimer or HER3/PI3K complex in the tumor sample,
wherein for measurement of HER2-HER3 heterodimer, the antibody composition binds specifically to HER3 and the cleaving probe binds specifically to HER2, or the antibody composition binds specifically to HER2 and the cleaving probe binds specifically to HER3, and wherein for measurement of HER3/PI3K complex, the antibody composition binds specifically to HER3 and the cleaving probe binds specifically to PI3K, or the antibody composition binds specifically to PI3K and the cleaving probe binds specifically to HER3.

13. The method of any one of claims 1-3, wherein measuring the amount of HER2-HER3 heterodimer in the tumor sample from the subject comprises the steps of:
a) contacting the tumor sample with a HER2 antibody composition;
b) contacting the tumor sample with a HER3 antibody composition;
c) contacting the tumor sample with a first binding composition that binds to either the HER2 antibody composition or the HER3 antibody composition, wherein the first binding composition comprises a molecular tag attached thereto via a cleavable linkage;
d) contacting the tumor sample with a cleaving probe, wherein the cleaving probe cleaves the cleavable linkage of the binding composition when within an effective proximity thereto;
e) cleaving the cleavable linker of the antibody composition, thereby releasing the molecular tag; and
f) quantitating the released molecular tag to determine the amount of HER2-HER3 heterodimer in the tumor sample,
wherein the cleaving probe binds specifically to HER2 if the antibody binding composition binds specifically to HER3, or the cleaving probe binds specifically to HER3 if the antibody binding composition binds specifically to HER2.

14. The method of any one of claims 1-3, wherein measuring the amount of HER3/PI3K complex in the tumor sample from the subject comprises the steps of:
a) contacting the tumor sample with a HER3 antibody composition and a PI3K antibody binding composition, wherein one antibody composition comprises a molecular tag attached thereto via a cleavable linkage and the other antibody composition comprises a cleaving probe that cleaves the cleavable linkage of the binding composition when within an effective proximity thereto;
b) cleaving the cleavable linker to release the molecular tag; and
c) quantitating the released molecular tag to determine the amount of HER3/PI3K complex in the tumor sample.

15. The method of any one of claims 1-3, wherein measuring the amount of phosphorylated HER3 in the tumor sample from the subject comprises the steps of:
a) contacting the tumor sample with a first HER3 antibody composition that specifically binds to HER3 protein at a first binding site, wherein the first HER3 antibody composition comprises a molecular tag attached thereto via a cleavable linkage;
b) contacting the tumor sample with a second HER3 antibody composition that specifically binds to HER3 protein at a second binding site, wherein the second HER3 antibody composition comprises a cleavage-inducing moiety that cleaves the cleavable linkage of the HER3 antibody composition when within an effective proximity thereto and wherein the second binding site comprises a HER3 phosphorylation site;
c) cleaving the cleavable linker of the first HER3 antibody composition, thereby releasing the molecular tag; and
d) quantitating the released molecular tag to determine the amount of phosphorylated HER3 in the tumor sample.

16. The method of claim 2 or 3, wherein the HER3-targeted therapy comprises at least one agent selected from the group consisting of U3-1289/AMG888, MM-121/SAR256212, MM-111, MEHD7945A, AZD-8931, LJM716, Av-203, and pertuzumab.

17. The method of claim 2, further comprising measuring the amount of total HER2 protein and determining if the subject has a HER2 positive cancer or a HER2 negative cancer.

18. The method of claim 2, further comprising determining if the amount of total HER2 protein is below a first cutoff comprising a level of total HER2 protein corresponding to the bottom 5^{th} percentile of total HER2 protein expression in a reference population of HER2 positive cancers, if the amount of total HER2 protein is above a second cutoff comprising a level of total HER2 protein corresponding to a top 95^{th} percentile of total HER2 protein expression in a reference population of HER2 negative cancers, or whether the amount of total HER2 protein is above the first cutoff but below the second cutoff.

19. The method of claim 17, further comprising administering a co-therapy comprising a HER2-targeted therapy and a HER3-targeted therapy if the subject has a HER2 positive cancer.

20. The method of claim 18, further comprising administering a co-therapy comprising a HER2-targeted therapy and a HER3-targeted therapy if the amount of total HER2 protein in the tumor sample is above the first cutoff.

21. The method of either claim 19 or 20, wherein the HER2 targeted therapy comprises at least one agent selected from the group consisting of BIBW 2992, HKI-272, 4D5, pertuzumab, trastuzumab, trastuzumab emtansine, AEE-788, lapatinib, neratinib, ARRY-380, and ARRY-543.

22. The method of claim 3, wherein responsiveness to a HER3-targeted agent comprises a longer disease time course between diagnosis and the occurrence of a significant event while the subject is being treated with a HER3-acting agent.

23. The method of claim 22, wherein the significant event comprises at least one of progression of the cancer from one stage to a more advanced stage, progression to metastatic disease, relapse, surgery, or death.

24. The method of claim 3, further comprising measuring the amount of total HER2 protein and determining if the subject has a HER2 positive cancer or a HER2 negative cancer.

25. The method of claim 3, further, comprising determining if the amount of total HER2 protein is below a first cutoff comprising a level of total HER2 protein corresponding to the bottom 5^{th} percentile of total HER2 protein expression in a reference population of HER2 positive cancers, if the amount of total HER2 protein is above a second cutoff comprising a level of total HER2 protein corresponding to a top 95^{th} percentile of total HER2 protein expression in a reference population of HER2 negative cancers, or whether the amount of total HER2 protein is above the first cutoff but below the second cutoff.

26. The method of claim 24, further comprising indicating that the subject is more likely to respond to a co-therapy of a HER2-targeted agent and a HER3-targeted agent if the subject has a HER2 positive cancer.

27. The method of claim 25, further comprising indicating that the subject is more likely to respond to a co-therapy of a HER2-targeted agent and a HER3-targeted agent if the amount of total HER2 protein in the biological sample is above the first cutoff.

28. The method of either claim 26 or 27, wherein the HER2-targeted therapy comprises at least one agent selected from the group consisting of BIBW 2992, HKI-272, 4D5, pertuzumab, trastuzumab, trastuzumab emtansine, AEE-788, lapatinib, neratinib, ARRY-380, and ARRY-543.
